(19) United States
(12) Patent Application Publication
(10) Pub. No.: US 2019/0093226 A1
(43) Pub. Date: Mar. 28, 2019

(54) METHOD FOR APPLYING A PECVD LUBRICITY LAYER WITH A MOVING GAS INLET

(71) Applicant: SIO2 MEDICAL PRODUCTS, INC., Auburn, AL (US)

(72) Inventors: James Newton, Auburn, AL (US); Brian Russell Lilly, Auburn, AL (US); Becky L. Clark, San Diego, CA (US)

(21) Appl. No.: 16/092,124

(22) PCT Filed: Apr. 7, 2017
(86) PCT No.: PCT/US2017/026575

§ 371 Date: Oct. 8, 2018

Related U.S. Application Data
(60) Provisional application No. 62/320,218, filed on Apr. 8, 2016.

(51) Int. Cl. C23C 16/507; C23C 16/40; C23C 16/455; A61M 5/31

(52) U.S. Cl. CPC C23C 16/507; C23C 16/401; C23C 16/45589; A61M 2207/10; A61M 2005/3131; A61M 2205/0222; A61M 2205/0238; A61M 5/3129

(57) ABSTRACT

A two-phase method is provided for applying a lubricity layer to a surface. The two-phase method comprises a low power deposition step and a high power crosslinking step. The method includes providing a surface of a vessel or an object to be processed. A gas inlet having an internal passage having at least one outlet is provided. An outer electrode is provided. A gaseous PECVD precursor is introduced via at least one outlet of the internal passage. Electromagnetic energy is applied to the outer electrode under conditions effective to form a PECVD lubricity layer on at least a portion of the inner surface. Relative axial motion between the vessel or the object and the gas inlet is provided during at least some time when electromagnetic energy is applied to the outer electrode.

### CROSS-REFERENCE TO RELATED APPLICATIONS

This specification claims the priority of U.S. Ser. Nos. 62/320,218 filed on Apr. 8, 2016. The entire application is incorporated by reference here to provide continuity of disclosure.

### FIELD OF INVENTION

The present invention relates to the technical field of fabrication of a surface (e.g. an interior surface of a syringe barrel, an exterior surface of a needle or a plunger), a plastic article, an object, a vessel, or a medical device part, having a lubricity coating applied by plasma enhanced chemical vapor deposition (PECVD). The invention also relates to fabrication of coated surface with an advantage of combination of high lubricity (e.g. low and consistent plunger force) and low particle count. The invention further relates to methods of applying a PECVD lubricity layer or coating to a surface, a plastic article, a vessel, a medical device part. More particularly, the invention relates to a coating method providing relative movement between a gas inlet and a coated substrate, when applying a lubricity layer to the surface of the substrate, using PECVD. The invention further relates to a two-phase coating method including a PECVD lubricity coating deposition applied using a first electromagnetic power at the first phase, followed by a crosslinking using a second electromagnetic power at the second phase, wherein the power level of the second power is higher than the power level of the first power.

### BACKGROUND

An important consideration for syringes or cartridges is to ensure that the plunger can move at a constant speed and with a constant force when it is pressed into the barrel. For this purpose, a lubricant, either on one or on both of the barrel and the plunger, is desirable. A similar consideration applies to vessels which have to be closed by a stopper, and to the stopper itself, and more generally to any surface which should provide a certain lubricity, for example, the exterior surface of a needle.

To reduce friction and thus improve plunger force, lubrication is traditionally applied to the barrel-contacting engagement surface of the plunger, the interior surface of the barrel, or both. Liquid or gel-like oily lubricants, such as free silicone oil (e.g., polydimethylsiloxane or “PDMS”), may provide a desired level of lubrication between the plunger and the barrel to optimize plunger force. PDMS is, in fact, a standard oily lubricant used in the industry.

However, one problem associated with oily lubricant is that an oily lubricant can mix and interact with the drug product in a vessel and they tend to generate more particulates or sub visible particulates which may also interact with the drug product held by the container. The oily lubricant and/or the particulates they generate can potentially degrade the drug or otherwise affect its efficacy and/or safety. Degradation is particularly an issue in the case of protein compositions and polypeptide compositions, which occupy a market with tremendous growth potential. Further, such lubricants may in some cases be problematic if they are injected into the patient along with the drug product.

Prefilled syringes are commonly prepared and sold so the syringe does not need to be filled before use. The syringes can be prefilled with saline solution, a dye for injection, or a pharmaceutically active preparation, for some examples. Besides the problematic issues mentioned in paragraph [0006], when used with prefilled syringes, the silicon oil particulates may migrate away from the plunger over time, resulting in spots between the plunger and the interior surface of the container with little or no lubrication. This may cause a phenomenon known as “stiction,” an industry term for the adhesion between the plunger and the barrel that needs to be overcome to break out the plunger and allow it to begin moving.

For these reasons, there is an industry need to achieve a combination of high lubricity (e.g. low and consistent break out and sliding forces) and low particle count for the surface of medical devices.

To avoid oily lubricants (i.e. to achieve “oil free” lubrication), U.S. Pat. No. 7,985,188 and EP2796591A1, which is incorporated herein by reference in its entirety, discloses methods and apparatuses for applying a lubricity layer to plastic containers using plasma enhanced chemical vapor deposition (“PECVD”). In one aspect, the method disclosed in that patent involves feeding a monomer into a vessel with a gas inlet tube when performing PECVD. The gas inlet tube, according to methods disclosed in that patent, is stationary during PECVD.

### SUMMARY

The present invention pertains to plastic vessels and medical devices, in particular vials and syringes, coated with PECVD coatings made from organosilicon precursors with further treatment. These novel devices offer the superior barrier properties of glass and the dimensional tolerances and breakage resistance of plastics, yet reduce or eliminate the drawbacks of both materials. In particular, a plasma coating (SiOₓC_{y}H_{z} or its equivalent Si_{w}OₓC_{y}H_{z}, in which x is from about 0.5 to about 2.4 as measured by X-ray photoelectron spectroscopy (XPS), y is from about 0.6 to about 3 as measured by XPS, and z is from about 2 to about 9 as measured by at least one of Rutherford backscattering spectrometry (RBS) or hydrogen forward scattering (HFS)) is provided. This plasma coating improves lubricity (“lubricity coating”), thus eliminating the need for traditional silicon oil lubricants e.g. in syringes. Further embodiments of the invention are methods to achieve high lubricity, low particle counts or combination of both by said coatings and the resulting coated devices.

The inventors have found that providing relative motion between the vessel and the gas inlet tube during PECVD provides high lubricity throughout the inner walls of the vessel and high electromagnetic power reduces sub-visible particulates from the lubricity layer in the vessel's liquid contents.

Accordingly, in one aspect, a method is provided for applying a lubricity layer to a surface of a vessel with a two-phase PECVD process. In at least one embodiment, the lubricity layer is applied to an inner surface of a vessel, such as a generally tubular vessel. The method includes providing a vessel, such as the generally tubular vessel, to be processed. The vessel may include an inner surface defining a lumen and an opening at an end of the vessel providing access to the lumen or an object to be processed including an outer surface. A gas inlet having an internal passage having at least one outlet is provided. An outer electrode is provided. At the first phase, a gaseous PECVD precursor is introduced into the lumen via at least one outlet of the internal passage. Electromagnetic energy is applied to the outer electrode under conditions effective to form a PECVD lubricity layer on at least a portion of the inner surface of the vessel. Relative axial motion between the vessel and the gas inlet is provided during at least some time when electromagnetic energy is applied to the outer electrode during the PECVD lubricity coating process, optionally at the first phase, or at the second phase or both. At the second phase, the PECVD lubricity coating deposited during the first phase is crosslinked with higher power level of electromagnetic energy than the power level of the electromagnetic energy applied at the first phase. The invention particularly relates to the PECVD lubricity coating process wherein the plasma-forming energy is applied at a first phase at a first energy level followed by a further treatment at a second phase at a second energy level higher than the first energy level. The invention particularly relates to a PECVD lubricity coating process which generates a lubricity coating with the advantage of a combination of consistent low plunger force and low sub-visible particle count.

The invention further pertains to a substrate coated with the product of the above method, to a vessel processing system for coating of a vessel, the system comprising a processing station arrangement configured for performing the above and/or below mentioned method steps.

The invention further pertains to a computer-readable medium, in which a computer program for coating of a vessel is stored which, when being executed by a processor of a vessel processing system, is adapted to instruct the processor to control the vessel processing system such that it carries out the above and/or below mentioned method steps.

The invention further pertains to a program element or computer program for coating of a vessel, which, when being executed by a processor of a vessel processing system, is adapted to instruct the processor to control the vessel processing system such that it carries out the above and/or below mentioned method steps.

The processor may thus be equipped to carry out exemplary embodiments of the methods of the present invention. The computer program may be written in any suitable programming language, for example, C++ and may be stored on the computer-readable medium, such as a CD-ROM. Also, the computer program may be available from a network, such as the Worldwide Web, from which it may be downloaded into image processing units or processors, or any suitable computers.

The current invention addresses the long-standing shortcomings of conventional silicone oil lubricant and the PECVD lubricity coatings currently in the art on parenteral syringes and cartridges. Specifically, a combination of consistent low plunger force and low sub-visible particle count is enabled by this invention through at least moving inlet and a post-deposition higher power crosslinking treatment.

There is a further need to optimize these factors while reducing manufacturing costs and complexity. The subject invention preferably addresses those needs, and others.

In the following paragraphs, coating methods and the coated devices resulting from the methods, in accordance with one or more embodiments of the present invention, are described. The methods can be carried out on the equipment (vessel processing system and vessel holder) which is also described below or in U.S. Pat. No. 7,985,188 and EP2796591A1, each of which is incorporated by reference herein in its entirety and for all purposes.

### BRIEF DESCRIPTION OF THE DRAWINGS

FIG. 1 is a schematic sectional view of a vessel holder in a coating station according to an embodiment of the disclosure.

FIG. 2 is a section taken along section lines A-A of FIG. 1.

FIG. 3 is a graph illustrating the effect of a moving gas inlet on high power OMCTS coating.

FIG. 4 is a graph illustrating the plunger force achieved with Process 1.

FIG. 5 is a graph illustrating the plunger force achieved with Process 2.

FIG. 6 is a graph illustrating the plunger force achieved with Process 3.

FIG. 7 is a graph illustrating the plunger force achieved with Process 4.

FIG. 8 is a graph illustrating the particle count in each size category achieved with Process 3.

FIG. 9 is a graph illustrating the particle count in each size category achieved with Process 4.

FIG. 10 shows the FT-IR spectrum for lubricity coating applied by Process 4.

FIGS. 11A and 11B show isometric and front views, respectively, of an exemplary holder (located at least partially within a coating chamber) useful to coat an outer surface of a vessel or an object, in accordance with at least one embodiment of the present invention.

FIG. 12 is a schematic sectional view of a syringe showing a coating set prepared on the substrate before a lubricity coating is applied according to an exemplary embodiment of the present invention.

FIG. 12A shows an enlarged detail view of the syringe barrel wall of FIG. 12.

FIG. 13 is a schematic sectional view of a syringe showing a barrier coating prepared on the substrate before a lubricity coating is applied according to an exemplary embodiment of the present invention.

FIG. 13A shows an enlarged detail view of the syringe barrel wall of FIG. 13.

FIG. 14 is a schematic sectional view of a syringe showing a schematic representation of a bilayer coating set (i.e. a tie layer and a barrier coating) prepared on the substrate before a lubricity coating is applied according to an exemplary embodiment of the present invention.

FIG. 14A shows an enlarged detail view of the syringe barrel wall of FIG. 14.

FIG. 15 is a schematic sectional view of a syringe showing a trilayer coating set (i.e. a tie layer, a barrier coating and a pH protective coating) prepared on the substrate before a lubricity coating is applied according to an exemplary embodiment of the present invention.

FIG. 15A shows an enlarged detail view of the syringe barrel wall of FIG. 15.

FIG. 16 is a schematic sectional view of a syringe showing a lubricity coating applied on a polymer substrate surface.

FIG. 16A shows an enlarged detail view of the syringe barrel wall of FIG. 16.

FIG. 17 is a schematic sectional view of a syringe showing a lubricity coating applied on a glass surface.

FIG. 17A shows an enlarged detail view of the syringe barrel wall of FIG. 17.

FIG. 18 is a sectional view of a syringe showing an exemplary depth of plunger insertion for plunger force testing.

The following reference characters are used in the drawing figures:

| | |
|---|---|
| 28 | Vessel processing system |
| 50 | Vessel holder |
| 70 | Conveyor |
| 80 | Vessel |
| 82 | Opening |
| 84 | Closed end |
| 86 | Wall |
| 88 | Interior surface |
| 90 | Coating(s) |
| 92 | Vessel port |
| 94 | Vacuum duct |
| 96 | Vacuum port |
| 98 | Vacuum source |
| 100 | O-ring (of 92) |
| 102 | O-ring (of 96) |
| 104 | Gas inlet port |
| 106 | O-ring (of 100) |
| 108 | Probe (inner electrode) |
| 110 | Gas delivery port (of 108) |
| 114 | Housing (of 50) |
| 116 | Collar |
| 118 | Exterior surface |
| 144 | PECVD gas source |
| 160 | Outer electrode |
| 162 | Power supply |
| 164 | Sidewall (of 160) |
| 166 | Sidewall (of 160) |
| 168 | Closed end (of 160) |
| 250 | Syringe barrel |
| 252 | Syringe |
| 254 | Interior surface (of 250) |
| 256 | Back end (of 250) |
| 258 | Plunger (of 252) |
| 260 | Front end (of 250) |
| 262 | Cap |
| 264 | Interior surface (of 262) |
| 268 | Vessel |
| 274 | Lumen |
| 280 | Polymer surface |
| 282 | Tie layer |
| 284 | pH protective layer |
| 285 | Underlying layer(s) |
| 286 | Lubricity layer |
| 288 | Barrier layer |
| 289 | Glass substrate |
| 500 | Object holder within a coating chamber |

### DETAILED DESCRIPTION OF THE PREFERRED EMBODIMENTS

The present invention will now be described more fully with reference to the accompanying drawings, in which several non-limiting embodiments are shown. This invention can, however, be embodied in many different forms and should not be construed as limited to the embodiments set forth here. Rather, these embodiments are examples of the invention, which has the full scope indicated by the language of the claims Like numbers refer to like or corresponding elements throughout.

A wide variety of different containers, vessels, or surfaces can be treated according to any embodiment. The terms “container” and “vessel” as used throughout this specification may be any type of article that is adapted to contain or convey a substrate. The substrate can be a liquid, a gas, a solid, or any two or more of these. One example of a container or vessel is an article with at least one opening (e.g., one, two or more, depending on the application) and a wall defining an interior contacting surface. One example of a surface is a vessel lumen surface, where the vessel is, for example, a vial, a tube, a bottle, ajar, a syringe, a cartridge, a blister package, a flexible package, or an ampoule. For more examples, the surface of the material can be a fluid surface of an article of labware, for example a microplate, a centrifuge tube, a pipette tip, a well plate, a microwell plate, an ELISA plate, a microtiter plate, a 96-well plate, a 384-well plate, a centrifuge tube, a chromatography vial, an evacuated blood collection tube, or a specimen tube, which are several non-limiting examples. The term “vessel” is used herein for simplicity to mean a container, a vessel, or a surface, unless otherwise specifically detailed in the appropriate context. One or two openings, like the openings of a sample tube (one opening) or a syringe barrel (two openings) are preferred. A vessel can be of any shape, a vessel having a substantially cylindrical wall adjacent to at least one of its open ends being preferred. Though the invention is not necessarily limited to vessels of a particular volume, vessels are contemplated in which the lumen has a void volume of from 0.5 to 50 mL, optionally from 1 to 10 mL, optionally from 0.5 to 5 mL, optionally from 1 to 3 mL. The substrate surface can be part or all of the inner surface of a vessel having at least one opening and an inner surface. Generally, the interior wall of the vessel is cylindrically shaped, like, e.g. in a sample tube or a syringe barrel. Sample tubes and syringes or cartridges or their parts (for example syringe or cartridge barrels) are contemplated.

An object can be of any shape with an outer surface to be processed. Optionally, the object can be a part of a medical device, for example, a piston, a plunger or a needle. The object can also be a vessel. When the outer surface of the object is to be processed, an object holder is utilized.

“Interior” and “inner” are exchangeable; and “exterior” and “outer” are exchangeable.

A crosslink is a bond formed between polymer chains. “Crosslinking” in the context of the present invention refers to any treatment uses crosslinks to modify the properties of the coating.

A “lubricity layer” or “lubricity coating” or any similar term is generally defined as a coating that reduces the frictional resistance of the coated surface, relative to the uncoated surface. In other words, it reduces the frictional resistance of the coated surface in comparison to a reference surface which is uncoated. The present lubricity layers are primarily defined by their lower frictional resistance than the uncoated surface and the process conditions providing lower frictional resistance than the uncoated surface. Optionally, the lubricity coating has a composition according to the empirical composition or sum formula Si_{w}OₓC_{y}H_{z}. It generally has an atomic ratio Si_{w}OₓC_{y}H_{z} wherein w is 1, x is from about 0.5 to about 2.4, y is from about 0.6 to about 3, preferably w is 1, x is from about 0.5 to 1.5, and y is from 0.9 to 2.0, more preferably w is 1, x is from 0.7 to 1.2 and y is from 0.9 to 2.0. The atomic ratio can be determined by XPS (X-ray photoelectron spectroscopy). Taking into account the H atoms, which are not measured by XPS, the coating may thus in one aspect have the formula Si_{w}OₓC_{y}H_{z}, or its equivalent SiOₓC_{y}H_{z}, for example where w is 1, x is from about 0.5 to about 2.4, y is from about 0.6 to about 3, and z is from about 2 to about 9 as measured by at least one of Rutherford backscattering spectrometry (RBS) and hydrogen forward scattering spectrometry (HFS), preferably RBS.

The values of w, x, y, and z used throughout this specification should be understood as ratios or an empirical formula (e.g. for a coating), rather than as a limit on the number or type of atoms in a molecule. For example, octamethylcyclotetrasiloxane, which has the molecular composition Si₄O₄C₈H₂₄, can be described by the following empirical formula, arrived at by dividing each of w, x, y, and z in the molecular formula by 4, the largest common factor: Si₁O₁C₂H₆. The values of w, x, y, and z are also not limited to integers. For example, (acyclic) octamethyltrisiloxane, molecular composition Si₃O₂C₈H₂₄, is reducible to Si₁O_{0.67}C_{2.67}H₈. Also, although SiOₓC_{y}H_{z} is described as equivalent to SiOₓC_{y}, it is not necessary to show the presence of hydrogen in any proportion to show the presence of SiOₓC_{y}.

“RF” is radio frequency.

“Fi” refers to the force required to initiate movement of the plunger rod or plunger tip within the barrel of a syringe, cartridge or any other vessel. “Fm” refers to the force required to maintain movement of the plunger rod or plunger tip within the barrel of a syringe, cartridge or any other vessel.

The term “at least” in the context of the present invention means “equal or more” than the integer following the term. The word “comprising” does not exclude other elements or steps, and the indefinite article “a” or “an” does not exclude a plurality unless indicated otherwise. Whenever a parameter range is indicated, it is intended to disclose the parameter values given as limits of the range and all values of the parameter falling within said range.

“First” and “second” or similar references to, e.g., processing stations or processing devices refer to the minimum number of processing stations or devices that are present, but do not necessarily represent the order or total number of processing stations and devices. These terms do not limit the number of processing stations or the particular processing carried out at the respective stations.

For purposes of the present invention, an “organosilicon precursor” is a compound having at least one of the linkage:

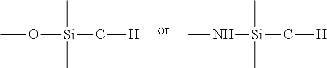

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a PECVD apparatus, is an optional organosilicon precursor. Optionally, the organosilicon precursor is selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors. For example, the organosilicon precursor can comprise octamethylenecyclotetrasiloxane (OMCTS), tetramethyldisiloxane (TMDSO), hexamethyldisiloxane (HMDSO), or a combination of two or more of these precursors.

A model representation of the lubricant molecular structure is shown below, which is an amorphous cross-linked organosiloxane chemistry.

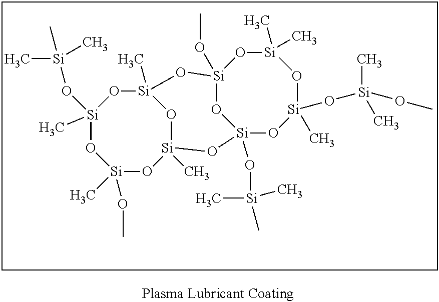

The feed amounts of PECVD precursors, gaseous reactant or process gases, and carrier gas are sometimes expressed in “standard volumes” in the specification and claims. The standard volume of a charge or other fixed amount of gas is the volume the fixed amount of the gas would occupy at a standard temperature and pressure (without regard to the actual temperature and pressure of delivery). Standard volumes can be measured using different units of volume, and still be within the scope of the present disclosure and claims. For example, the same fixed amount of gas could be expressed as the number of standard cubic centimeters, the number of standard cubic meters, or the number of standard cubic feet. Standard volumes can also be defined using different standard temperatures and pressures, and still be within the scope of the present disclosure and claims. For example, the standard temperature might be 0° C. and the standard pressure might be 760 Torr (as is conventional), or the standard temperature might be 20° C. and the standard pressure might be 1 Torr. But whatever standard is used in a given case, when comparing relative amounts of two or more different gases without specifying particular parameters, the same units of volume, standard temperature, and standard pressure are to be used relative to each gas, unless otherwise indicated.

The corresponding feed rates of PECVD precursors, gaseous reactant or process gases, and carrier gas are expressed in standard volumes per unit of time in the specification. For example, in the working examples the flow rates are expressed as standard cubic centimeters per minute, abbreviated as sccm. As with the other parameters, other units of time can be used, such as seconds or hours, but consistent parameters are to be used when comparing the flow rates of two or more gases, unless otherwise indicated.

“Frictional resistance” can be static frictional resistance and/or kinetic frictional resistance.

The “plunger sliding force” (synonym to “glide force,” “maintenance force,” Fₘ, which may also be used in this description) in the context of the present invention is the force required to maintain movement of a plunger in a syringe barrel, e.g. during aspiration or dispense. It can advantageously be determined using the ISO 7886-1:1993 test known in the art. A synonym for “plunger sliding force” often used in the art is “plunger force” or “pushing force”.

The “plunger breakout force” (synonym to “breakout force”, “break loose force”, “initiation force”, Fᵢ, which may also be used in this description) in the context of the present invention is the initial force required to move the plunger in a syringe, for example in a prefilled syringe.

Sliding force and breakout force may sometimes be used herein to describe the forces required to advance a stopper or other closure into a vessel, such as a medical sample tube or a vial, to seat the stopper in a vessel to close the vessel. Its use is analogous to use in the context of a syringe and its plunger, and the measurement of these forces for a vessel and its closure are contemplated to be analogous to the measurement of these forces for a syringe, except that at least in most cases no liquid is ejected from a vessel when advancing the closure to a seated position.

The plunger sliding force as defined and determined herein is suitable to determine the presence or absence and the lubricity characteristics of a lubricity layer or coating in the context of the present invention whenever the coating is applied to any syringe or syringe part, for example to the inner wall of a syringe barrel. The breakout force is of particular relevance for evaluation of the coating effect on a prefilled syringe, i.e. a syringe which is filled after coating and can be stored for some time, e.g. several months or even years, before the plunger is moved again (has to be “broken out”).

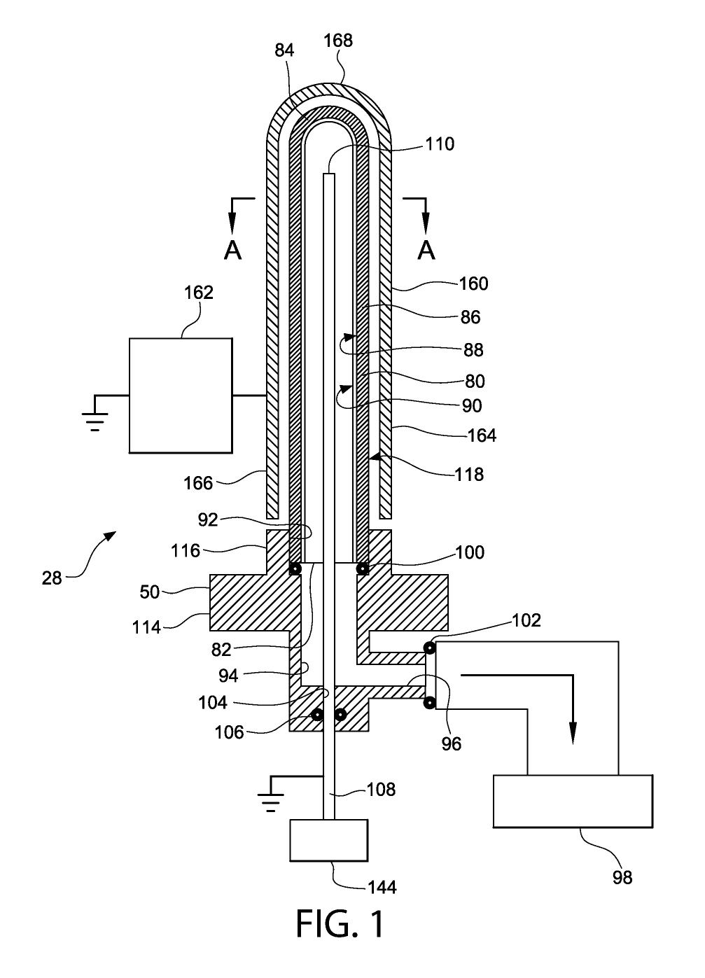

An exemplary vessel holder 50 is shown in FIG. 1. The vessel holder 50 has a vessel port 82 configured to receive and seat the opening of a vessel 80. The interior surface of a seated vessel 80 can be processed via the vessel port 82. The vessel holder 50 can include a duct, for example a vacuum duct 94, for withdrawing a gas from a vessel 80 seated on the vessel port 92. The vessel holder can include a second port, for example a vacuum port 96 communicating between the vacuum duct 94 and an outside source of vacuum, such as the vacuum pump 98. The vessel port 92 and vacuum port 96 can have sealing elements, for example O-ring butt seals, respectively 100 and 102, or side seals between an inner or outer cylindrical wall of the vessel port 82 and an inner or outer cylindrical wall of the vessel 80 to receive and form a seal with the vessel 80 or outside source of vacuum 98 while allowing communication through the port. Gaskets or other sealing arrangements can or also be used.

FIG. 1 also illustrates that the vessel holder, for example 50, can have a collar 116 for centering the vessel 80 when it is approaching or seated on the port 92.

In the embodiment illustrated in FIG. 1, the vessel holder 50 comprises a gas inlet port 104 for conveying a gas into a vessel seated on the vessel port. The gas inlet port 104 has a sliding seal provided by at least one O-ring 106, or two O-rings in series, or three O-rings in series, which can seat against a cylindrical probe 108 when the probe 108 is inserted through the gas inlet port 104. The probe 108 can be a gas inlet conduit that extends to a gas delivery port at its distal end 110. The distal end 110 of the illustrated embodiment can be inserted deep into the vessel 80 for providing one or more PECVD reactants and other process gases.

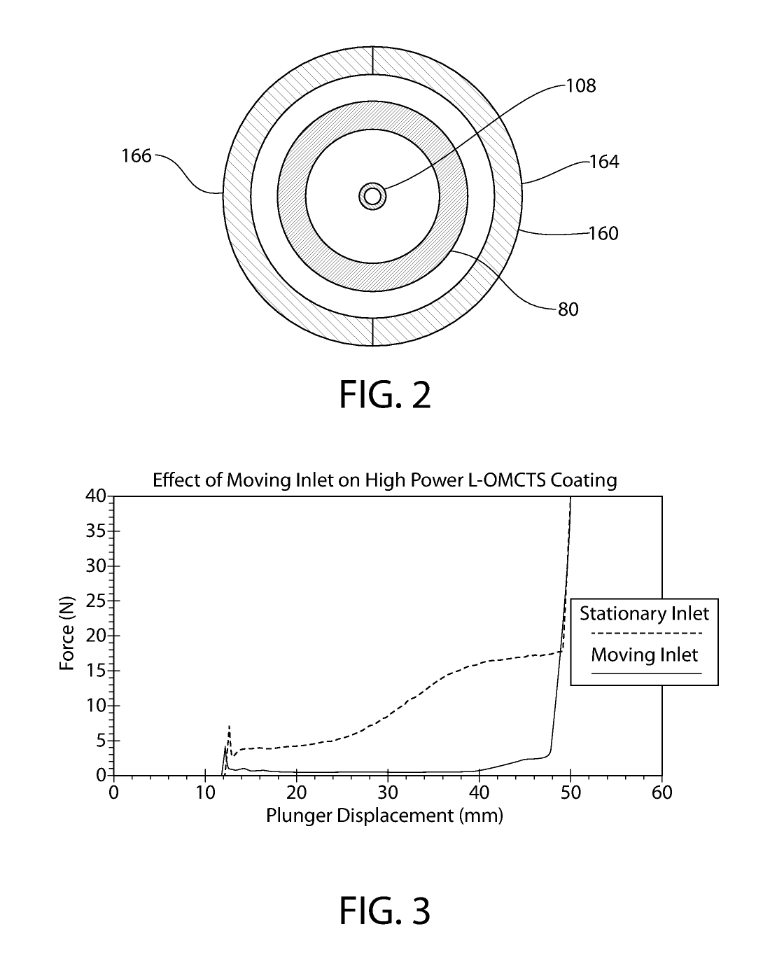

Referring to FIGS. 1 and 2, the processing station 28 can include an electrode 160 fed by a radio frequency power supply 162 for providing an electric field for generating plasma within the vessel 80 during processing. In this embodiment, the probe 108 is also electrically conductive and is grounded, thus providing a counter-electrode within the vessel 80. Alternatively, in any embodiment the outer electrode 160 can be grounded and the probe 108 directly connected to the power supply 162. Alternatively in any embodiment, microwave power can be used to provide the electric field for generating plasma.

A PECVD apparatus may include a vessel holder or an object holder, an inner electrode, an outer electrode, and a power supply. Optionally a vessel seated on the vessel holder defines a plasma reaction chamber, which optionally can be a vacuum chamber. Optionally the object with an outer surface to be processed is placed at least partially within a chamber. Optionally, a source of vacuum, a reactant gas source, a gas feed or a combination of two or more of these can be supplied. Optionally, a gas drain, not necessarily including a source of vacuum, is provided to transfer gas to or from the interior of a vessel seated on the port to define a closed chamber.

The PECVD apparatus can be used for atmospheric-pressure PECVD, in which case the plasma reaction chamber does not need to function as a vacuum chamber.

One of the optional embodiments of the present invention is a syringe part, e.g. a syringe barrel or plunger, coated with a lubricity layer.

Apparatus and general conditions suitable for carrying out this method are described in U.S. Pat. No. 7,985,188 and EP2796591A1, which is incorporated here by reference in full.

A general PECVD lubricity coating method includes providing a gas including an organosilicon precursor, optionally an oxidizing gas (for example O₂), and an inert gas in the vicinity of the substrate surface. The inert gas optionally is a noble gas, for example argon, helium, krypton, xenon, neon, or a combination of two or more of these inert gases. Plasma is generated in the gas by providing plasma-forming energy adjacent to the plastic substrate. As a result, a lubricity coating or layer is formed on the substrate surface by plasma enhanced chemical vapor deposition (PECVD).

A drawback of some lubricity coatings in the prior art is that due to the low crosslinking nature of lubricity coating, many small spherical ‘droplets’ can be liberated from the coating on the inside of the container if it is partially filled with water and shaken.

The approach of the current specification to solving these issues is a lubricity layer that is chemically attached to the syringe barrel wall and moderately cross-linked to limit its mobility. The lubricity coating, therefore, remains stationary with no migration on the surface and minimal particle count.

Increasing the RF power was found to produce a coating that is more strongly adhered to the container, however the lubricity of the resultant coating was found to be localized to the region close to the location of the gas inlet during the deposition. Plunger tests show that the gliding force for the plunger is good in the region near the location of the gas inlet, but the gliding force increases to unacceptable levels as the plunger moves away from that position (see Example 4).

It was found that moving the gas inlet axially along the direction from the flange to the needle during the deposition has the effect of extending the lubricity of the coating along a greater portion of the container. Therefore, combination of moving inlet at the first phase (i.e. deposition) and increasing the power level at the second phase (crosslinking) allows the production of a better coating that has less particulate formation while maintains low plunger force throughout the entire length of the container.

The plunger sliding force test is a specialized test of the coefficient of sliding friction of the plunger within a syringe, accounting for the fact that the normal force associated with a coefficient of sliding friction as usually measured on a flat surface is addressed by standardizing the fit between the plunger or other sliding element and the tube or other vessel within which it slides. The parallel force associated with a coefficient of sliding friction as usually measured is comparable to the plunger sliding force measured as described in this specification. Plunger sliding force can be measured, for example, as provided in the ISO 7886-1:1993 test.

In another aspect, the invention involves applying a lubricity layer derived from an organosilicon precursor. A lubricity layer optionally has a composition according to the empirical composition Si_{w}OₓC_{y} or Si_{w}NₓC_{y}, where w is 1, x is from about 0.5 to 2.4, and y is from about 0.6 to about 3. If the coated object is a syringe (or syringe part, e.g. syringe barrel) or any other item generally containing a plunger or movable part in sliding contact with the coated surface, the frictional resistance has two main aspects—breakout force and plunger sliding force.

In the context of the present invention, the following PECVD method is generally applied, which contains the following steps: (a) providing a gaseous reactant comprising a precursor as defined herein, optionally an organosilicon precursor, and optionally O₂ in the vicinity of the substrate surface; (b) generating plasma from the gaseous reactant, thus forming a coating on the substrate surface by plasma enhanced chemical vapor deposition (PECVD), wherein the plasma-forming energy is applied in a first phase at a first energy level; and c) further treating by plasma at a second phase at a second energy level higher than the first energy level, optionally in the absence of organosiloxane precursors. The treatment of plasma at a second phase at a second energy made occur continuously or following an interval after the first phase at a first energy level. Similarly, the second phase at a second energy may occur at the same apparatus, stage, or step; or at a different apparatus, stage, or step of the method and processing equipment as the first phase at a first energy level.

In the method, the coating characteristics are advantageously set by one or more of the following conditions: the plasma properties, the pressure under which the plasma is applied, the power applied to generate the plasma, the presence and relative amount of O₂ in the gaseous reactant, the plasma volume, and the organosilicon precursor. Optionally, the coating characteristics are set by the presence and relative amount of O₂ in the gaseous reactant and/or the power applied to generate the plasma.

In all embodiments of the present invention, the plasma is in an optional aspect hollow cathode plasma. In a further preferred aspect, the plasma is generated at reduced pressure (as compared to the ambient or atmospheric pressure). Optionally, the reduced pressure is less than 300 mTorr, optionally less than 200 mTorr, even optionally less than 100 mTorr.

The PECVD optionally is performed by energizing the gaseous reactant containing the precursor with electrodes powered at a frequency at microwave or radio frequency, and preferably at a radio frequency. The radio frequency preferred to perform an embodiment of the invention will also be addressed as “RF frequency”. A typical radio frequency range for performing the present invention is a frequency of from 10 kHz to less than 300 MHz, optionally from 1 to 50 MHz, even optionally from 10 to 15 MHz. A frequency of 13.56 MHz is most preferred, this being a government sanctioned frequency for conducting PECVD work.

For any coating of the present invention, the plasma is generated with electrodes powered with sufficient power to form a coating on the substrate surface. For a lubricity coating of the present invention, in the method according to an embodiment of the invention, the plasma is optionally generated at the first phase to deposit a lubricity coating (i) with electrodes supplied with an electric power of from 0.1 to 50 W, optionally from 2 to 30 W, optionally from 2 to 20 W, preferably 4 to 10 W, for example of 5 W; and/or (ii) wherein the ratio of the electrode power to the plasma volume is less than 10 W/ml, optionally is from 5 W/ml to 0.1 W/ml, optionally is from 4 W/ml to 0.1 W/ml, optionally from 2 W/ml to 0.2 W/ml.

The power (in Watts) used for PECVD also has an influence on the coating properties. Typically, an increase of the power will increase the barrier properties of the coating, and a decrease of the power will increase the lubricity and hydrophobicity of the coating. E.g., for a coating on the inner wall of syringe barrel having a volume of about 3 ml, a power of less than 30 W will lead to a coating which is predominantly a lubricity coating, while a power of more than 30 W will lead to a coating which is predominantly a barrier layer.

A further parameter determining the coating properties is the ratio of O₂ (or another oxidizing agent) to the precursor (e.g. organosilicon precursor) in the gaseous reactant used for generating the plasma. Typically, an increase of the O₂ ratio in the gaseous reactant will increase the barrier properties of the coating, and a decrease of the O₂ ratio will increase the lubricity and hydrophobicity of the coating.

If a lubricity layer is desired, then O₂ is optionally present in a volume-volume ratio to the gaseous reactant of from 0:1 to 5:1, optionally from 0:1 to 1:1, even optionally from 0:1 to 0.5:1 or even from 0:1 to 0.1:1. Most advantageously, essentially no oxygen is present in the gaseous reactant. Thus, the gaseous reactant should comprise less than 1 vol % O₂, for example less than 0.5 vol % O₂, and optionally is O₂-free. The same applies to a hydrophobic layer.

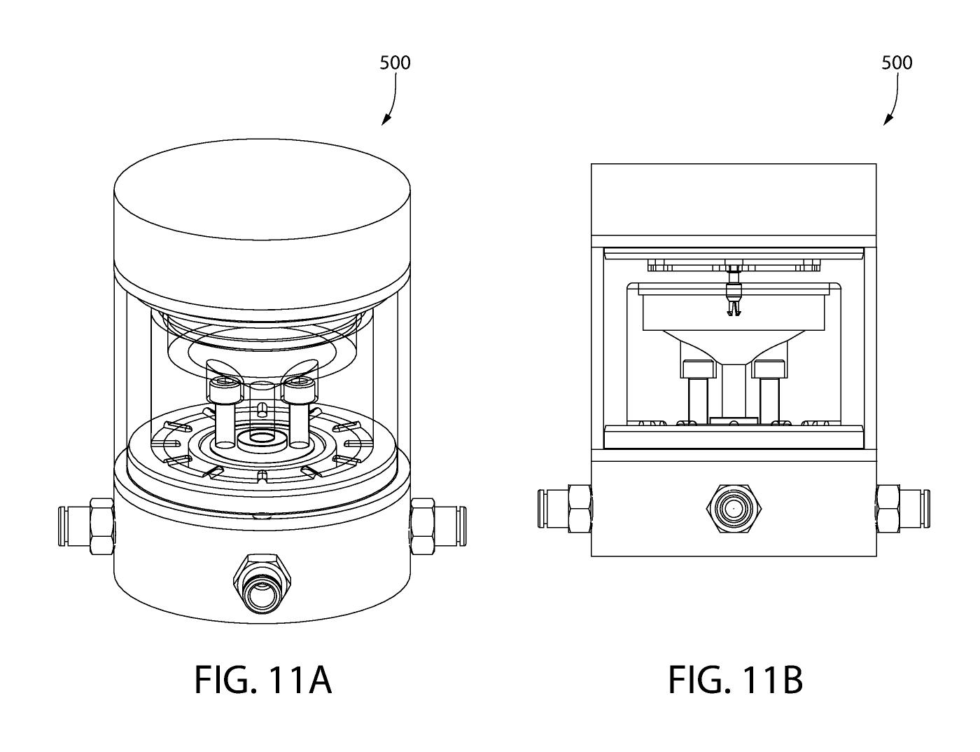

The two-phase PECVD process of the present invention may be utilized to coat or treat outer surfaces, in accordance with at least one embodiment of the present invention, such as the outer surface of an object. The object can also include a vessel. For example, the two-phase PECVD process may be utilized to coat the outer surface of a vessel, such as vial, syringe, cartridge, multi-well plate, bag, or tube. Similarly, the two-phase PECVD process may be utilized to coat a surface, such as the outer surface, of an object for such products, such as the outer surface of a plunger, stopper, or seal. For such applications, the coating process may utilize a product or object holder or fixture to enable the coating or treatment of the desired surface(s). One exemplary holder within a coating chamber 500 useful to coat a surface of a vessel or object, in accordance with at least one embodiment of the present invention, shown in FIGS. 11A and 11B. FIG. 11A shows an isometric view of the object holder within the chamber 500, while FIG. 11B shows a front view of the object holder within the chamber 500. In this configuration, the holder within the chamber 500 may be utilized to coat or treat a surface of an object, such as coating an outer surface of a plunger useful in syringes. Such syringe may optionally contain the same coating, enabled by the two-phase PECVD process of the present invention, on its interior surface; thereby enabling the surface interaction between the plunger and the syringe to have the same coating. Other such holders within a chamber can similarly be utilized to enable coating of various interior or exterior surfaces of vessels and objects, as would be readily appreciated by one having ordinary skill in the art of holders or fixtures for processing of vessels and objects.

### Vessel

In any embodiment, the substrate optionally can be injection molded, blow molded, or otherwise formed from a polymer selected from the group consisting of a polycarbonate, an olefin polymer, for example a cyclic olefin copolymer (COC), a cyclic olefin polymer (COP), or polypropylene, and preferably COP; a polyester, for example polyethylene terephthalate or polyethylene naphthalate; polylactic acid; a polymethylmethacrylate; or a combination of any two or more of these.

Some utilities of coating a vessel in whole or in part with a lubricity layer, such as selectively at surfaces contacted in sliding relation to other parts, is to ease the insertion or removal of a stopper or passage of a sliding element such as a piston in a syringe or a stopper in a sample tube. The vessel can be made of glass or a polymer material such as polyester, for example polyethylene terephthalate (PET), a cyclic olefin copolymer (COC), an olefin such as polypropylene, or other materials. Applying a lubricity layer by PECVD can avoid or reduce the need to coat the vessel wall or closure with a sprayed, dipped, or otherwise applied organosilicon or other lubricant that commonly is applied in a far larger quantity than would be deposited by a PECVD process.

The vessel 80 can also be made, for example of glass of any type used in medical or laboratory applications, such as soda-lime glass, borosilicate glass, or other glass formulations. Other vessels having any shape or size, made of any material, are also contemplated for use in the system. One function of coating a glass vessel can be to reduce the ingress of ions in the glass, either intentionally or as impurities, for example sodium, calcium, or others, from the glass to the contents of the vessel, such as a reagent or blood in an evacuated blood collection tube. Another function of coating a glass vessel in whole or in part, such as selectively at surfaces contacted in sliding relation to other parts, is to provide lubricity to the coating, for example to ease the insertion or removal of a stopper or passage of a sliding element such as a piston in a syringe. Still another reason to coat a glass vessel is to prevent a reagent or intended sample for the vessel, such as blood, from sticking to the wall of the vessel or an increase in the rate of coagulation of the blood in contact with the wall of the vessel.

A related embodiment is a vessel made of plastic, in which the barrier coating is made of soda lime glass, borosilicate glass, or another type of glass.

In any embodiment, the container size optionally can be from 0.5 to 10 mL, alternatively from 1 to 3 mL, alternatively from 6 to 10 mL.

One optional type of vessel 268 is a syringe including a plunger, a syringe barrel, and a lubricity coating as defined above on either one or both of these syringe parts, preferably on the inside wall of the syringe barrel. The syringe barrel includes a barrel having an interior surface slidably receiving the plunger. The lubricity coating may be disposed on the interior surface of the syringe barrel, or on the plunger surface contacting the barrel, or on both surfaces. The lubricity coating is effective to reduce the breakout force or the plunger sliding force necessary to move the plunger within the barrel.

The syringe optionally further includes a staked needle. The needle is hollow with a typical size ranging from 18-29 gauge. The syringe barrel has an interior surface slidably receiving the plunger. The staked needle may be affixed to the syringe during the injection molding of the syringe or may be assembled to the formed syringe using an adhesive. A cover is placed over the staked needle to seal the syringe assembly. The syringe assembly must be sealed so that a vacuum can be maintained within the syringe to enable the PECVD coating process.

As another option, the syringe can comprise a Luer fitting at its front end. The syringe barrel has an interior surface slidably receiving the plunger. The Luer fitting includes a Luer taper having an internal passage defined by an internal surface. The Luer fitting optionally can be formed as a separate piece from the syringe barrel and joined to the syringe barrel by a coupling.

Another aspect of the invention is a plunger for a syringe, including a piston, and a push rod. The piston has a front face, a generally cylindrical side face, and a back portion, the side face being configured to movably seat within a syringe barrel. The plunger has a lubricity coating according to the present invention on its side face. The push rod engages the back portion of the piston and is configured for advancing the piston in a syringe barrel. The plunger may additionally comprise a SiOₓ barrier coating, in which x is from 1.5 to 2.9 as measured by XPS

A further aspect of the invention is a vessel with just one opening, which can be, for example, a vessel for collecting or storing a compound or composition. Such vessel is in a specific aspect a tube, e.g. a sample collecting tube, e.g., a blood collecting tube. Such a tube may be closed with a closure, e.g. a cap or stopper. Such cap or stopper may comprise a lubricity coating according to the present invention on its surface which is in contact with the tube, and/or it may contain a passivating coating according to the present invention on its surface facing the lumen of the tube. In a specific aspect, such a stopper or a part thereof may be made from an elastomeric material.

A further particular aspect of the invention is a syringe barrel coated with the lubricity coating as defined in the preceding paragraph.

### First Phase of Gas Feed to PECVD Apparatus for Depositing Lubricity Coating

A precursor is included in the gas feed provided to the PECVD apparatus. Preferably, the precursor is an organosilicon compound (in the following also designated as “organosilicon precursor”), more preferably an organosilicon compound selected from the group consisting of a linear siloxane, a monocyclic siloxane, a polycyclic siloxane, a polysilsesquioxane, an alkyl trimethoxysilane, an aza analogue of any of these precursors (i.e. a linear silazane, a monocyclic silazane, a polycyclic silazane, a polysilsesquioxazane), and a combination of any two or more of these precursors. The precursor is applied to a substrate under conditions effective to form a coating by PECVD. The precursor is thus polymerized, crosslinked, partially or fully oxidized, or any combination of these. In any embodiment, the organosilicon precursor optionally can include a linear or monocyclic siloxane, optionally comprising or consisting essentially of octamethylcyclotetrasiloxane (OMCTS), tetramethylcyclotetrasiloxane (TMCTS), hexamethyldisiloxane (HMDSO), tetramethyldisiloxane (TMDSO), or a combination of two or more of these.

The oxidizing gas, optionally included in the gas feed, can comprise or consist of air, oxygen (O₂ and/or O₃, the latter commonly known as ozone), nitrous oxide, or any other gas that oxidizes the precursor during PECVD at the conditions employed. The oxidizing gas comprises about 1 standard volume of oxygen. The gaseous reactant or process gas can be at least substantially free of nitrogen. In any embodiment. O₂ optionally can be present, preferably in a volume-volume ratio to the organosilicon precursor of from 0:1 to 2:1, optionally from 0:1 to 0.5:1, optionally from 0.01:1 to 1:1, optionally from 0.01:1 to 0.5:1, optionally from 0.1:1 to 1:1.

In any embodiment, Ar optionally can be present as the inert gas. The gas optionally can be from 1 to 6 standard volumes of the organosilicon precursor, from 1 to 100 standard volumes of the inert gas, and from 0.1 to 2 standard volumes of O₂. In any embodiment, both Ar and O₂ optionally can be present.

The method of the invention may comprise the application of one or more coatings made by PECVD from the same or different organosilicon precursors under the same or different reaction conditions. E.g. a syringe may first be coated with an SiOₓ barrier coating using HMDSO as organosilicon precursor, and subsequently with a lubricity coating using OMCTS as organosilicon precursor. OMCTS is one of the highest molecular weight and boiling point organosiloxanes that can still be vaporized and delivered inside the syringe under a partial vacuum.

The extent of cross-linking is critical to balancing plunger force and subvisible particulates. Too much cross-linking results in a dense coating free of low molecular weight siloxane oligomers and oil droplets, but has no lubrication characteristics. Too little cross-linking results in a loosely networked oil with excellent lubrication, but many siloxane oligomers that can form sub-visible droplets. The extent of cross-link density of the plasma lubricant can be indirectly characterized by Fourier Transform infra-red (FTIR).

A gaseous reactant or process gas can be employed having a standard volume ratio of, for example when a lubricity coating is prepared:
from 1 to 6 standard volumes, optionally from 2 to 4 standard volumes, optionally equal to or less than 6 standard volumes, optionally equal to or less than 2.5 standard volumes, optionally equal to or less than 1.5 standard volumes, optionally equal to or less than 1.25 standard volumes of the precursor;
from 1 to 100 standard volumes, optionally from 5 to 100 standard volumes, optionally from 10 to 70 standard volumes, of a carrier gas;
from 0.1 to 2 standard volumes, optionally from 0.2 to 1.5 standard volumes, optionally from 0.2 to 1 standard volumes, optionally from 0.5 to 1.5 standard volumes, optionally from 0.8 to 1.2 standard volumes of an oxidizing agent.

### Second Phase of Gas Feed to PECVD Apparatus for Crosslinking

Optionally the gas feed comprises air, oxygen, nitrogen, carbon dioxide, ozone, hydrogen peroxide, any noble gas or any combination of two or more of the above.

Optionally the gas feed comprises the gases in absence of organosiloxane precursors in the second phase. This is a crosslinking step wherein the lubricity coating deposited in the first phase is treated with higher electromagnetic power without deposition of coating in the second phase. Therefore, no organosiloxane precursors are needed in this phase.

### Process Pressure for Depositing Lubricity Coating

PECVD may be carried out at any suitable pressure or vacuum level. For example, the process pressure optionally can be from 0.001 to 100 Torr (from 0.13 Pa to 13,000 Pa), optionally from 0.01 to 10 Torr (from 1.3 to 1300 Pa), optionally from 0.1 to 10 Torr (from 13 to 1300 Pa).

### First Phase of Plasma Forming Energy for Depositing Lubricity Coating

In any embodiment, the plasma optionally can be generated with microwave energy or RF energy. The plasma optionally can be generated with electrodes powered at a radio frequency, preferably at a frequency of from 10 kHz to less than 300 MHz, more preferably of from 1 to 50 MHz, even more preferably of from 10 to 15 MHz, most preferably at 13.56 MHz.

In any embodiment, the ratio of the electrode power to the plasma volume for the first pulse optionally can be equal to or more than 1 W/ml, preferably is from 2 W/ml to 50 W/ml, more preferably is from 3 W/ml to 10 W/ml.

In any embodiment, the first pulse optionally can be applied for 0.1 to 5 seconds, alternatively 0.5 to 3 seconds, alternatively 0.75 to 1.5 seconds. The first phase energy level optionally can be applied in at least two pulses.

### Second Phase of Plasma Forming Energy for Crosslinking

In any embodiment, the second phase electromagnetic energy applied to crosslink the lubricity layer which is deposited in the first phase is higher than the energy in the first phase. The energy level in the second phase optionally can be 5 W to 100 W, preferably 20 W to 70 W, most preferably 30 W to 50 W.

### Relation Between First and Second Phases Electromagnetic Power

In any embodiment, the plasma-forming energy optionally can be applied in the first phase at a first energy level, followed by further treatment in a second phase at a second energy level. The second energy level is higher than the first energy level.

### Additional PECVD Coating or Treatment

The vessel can be, for example, a syringe barrel, cartridge, or vial. The vessel has a thermoplastic wall having an interior surface enclosing at least a portion of the lumen, an exterior surface, and a coating set on at least one of the interior surface and the exterior surface of the wall. The coating set can include at least one of a tie coating or layer, a barrier coating or layer or a pH protective coating or layer, and a lubricity coating or layer of the current invention. Some embodiments of the present invention relate to the methods to prepare such coating set on the substrate.

The tie coating or layer can be formed on the interior surface or the exterior surface. It has the composition SiOₓC_{y}H_{z} in which x is from about 0.5 to about 2.4 as measured by X-ray photoelectron spectroscopy (XPS), y is from about 0.6 to about 3 as measured by XPS, and z is from about 2 to about 9 as measured by at least one of Rutherford backscattering spectrometry (RBS) or hydrogen forward scattering (HFS). The tie coating or layer has a facing surface facing toward the wall, and an opposed surface facing away from the wall.

The barrier coating or layer has the composition SiOₓ, in which x is from about 1.5 to about 2.9 as measured by XPS. The barrier coating or layer has a facing surface facing toward the opposed surface of the tie coating or layer (if present) and an opposed surface facing away from the tie coating or layer (if present).

The pH protective coating or layer, if present, has the composition SiOₓC_{y}H_{z}, in which x is from about 0.5 to about 2.4 as measured by XPS, y is from about 0.6 to about 3 as measured by XPS, and z is from about 2 to about 9 as measured by at least one of RBS or HFS. The pH protective coating or layer, if present, has a facing surface facing toward the opposed surface of the barrier layer (if present) and an opposed surface facing away from the barrier layer (if present).

In any embodiment, the contemplated method optionally includes a step for preparing a barrier coating or other optional coating set 285 on the substrate before the lubricity coating 286 is applied. The additional step optionally includes introducing a gas comprising an organosilicon precursor and O₂ in the vicinity of the substrate surface and generating plasma from the gas, thus forming an SiOₓ barrier coating on the substrate surface by plasma enhanced chemical vapor deposition (PECVD). Optionally, in the step for preparing a barrier coating, the plasma can be generated with electrodes powered with sufficient power to form a SiOₓ barrier coating on the substrate surface. The electrodes optionally are supplied with an electric power of from 8 to 500 W, preferably from 20 to 400 W, more preferably from 35 to 350 W, even more preferably of from 44 to 300 W, most preferably of from 44 to 70 W. In any embodiment of barrier coating, the O₂ optionally can be present in a volume:volume ratio of from 1:1 to 100:1 in relation to the silicon containing precursor, preferably in a ratio of from 5:1 to 30:1, more preferably in a ratio of from 10:1 to 20:1, even more preferably in a ratio of 15:1.

Optionally, the lubricity layer of the current invention is applied on a surface coated using organosiloxane as the gas precursor.

Another embodiment contemplated here, for adjacent layers of SiOₓ and a lubricity layer or coating and/or hydrophobic layer, is a graded composite of Si_{w}OₓC_{y}H_{z}. A graded composite can be separate layers of a lubricity layer or coating and/or hydrophobic layer or coating and SiOₓ with a transition or interface of intermediate composition between them, or separate layers of a lubricity layer or coating and/or hydrophobic layer or coating and SiOₓ with an intermediate distinct layer or coating of intermediate composition between them, or a single layer or coating that changes continuously or in steps from a composition of a lubricity layer or coating and/or hydrophobic layer or coating to a composition more like SiOₓ, going through the coating in a normal direction.

The grade in the graded composite can go in either direction. For example, a lubricity layer or coating and/or hydrophobic layer or coating can be applied directly to the substrate and graduate to a composition further from the surface of SiOₓ. Or, the composition of SiOₓ can be applied directly to the substrate and graduate to a composition further from the surface of a lubricity layer or coating and/or hydrophobic layer. A graduated coating is particularly contemplated if a coating of one composition is better for adhering to the substrate than the other, in which case the better-adhering composition can, for example, be applied directly to the substrate. It is contemplated that the more distant portions of the graded coating can be less compatible with the substrate than the adjacent portions of the graded coating, since at any point the coating is changing gradually in properties, so adjacent portions at nearly the same depth of the coating have nearly identical composition, and more widely physically separated portions at substantially different depths can have more diverse properties. It is also contemplated that a coating portion that forms a better barrier against transfer of material to or from the substrate can be directly against the substrate, to prevent the more remote coating portion that forms a poorer barrier from being contaminated with the material intended to be barred or impeded by the barrier.

The coating, instead of being graded, optionally can have sharp transitions between one layer or coating and the next, without a substantial gradient of composition. Such coatings can be made, for example, by providing the gases to produce a layer or coating as a steady state flow in a non-plasma state, then energizing the system with a brief plasma discharge to form a coating on the substrate. If a subsequent coating is to be applied, the gases for the previous coating are cleared out and the gases for the next coating are applied in a steady-state fashion before energizing the plasma and again forming a distinct layer or coating on the surface of the substrate or its outermost previous coating, with little if any gradual transition at the interface.

Optionally, after the lubricity layer or coating is applied, it can be post-cured after the PECVD process. Radiation curing approaches, including UV-initiated (free radial or cationic), electron-beam (E-beam), and thermal as described in Development Of Novel Cycloaliphatic Siloxanes For Thermal And UV-Curable Applications (Ruby Chakraborty Dissertation, can 2008) be utilized. For example, in any embodiment, the lubricity coated container optionally can be further treated by post heating it, optionally at 50 to 110 degrees C., alternatively 80 to 100 degree C., optionally for a time interval of 1 to 72 hours, alternatively 4 to 48 hours, alternatively 8 to 32 hours, alternatively 20 to 30 hours. In any embodiment, the post heating step optionally can be carried out under at least partial vacuum, alternatively under a pressure of less than 50 Torr, alternatively under a pressure of less than 10 Torr, optionally under a pressure of less than 5 Torr, alternatively at a pressure of less than 1 Torr.

### Properties

In any embodiment, the result of the present treatment can be a coated substrate coated with a lubricity coating. The lubricity coating optionally can have an average thickness of from 1 to 5000 nm, alternatively from 30 to 1000 nm, as another alternative from 100 to 500 nm. The optional SiOₓ barrier coating or layer of any embodiment optionally can have a thickness of from 20 to 30 nm.

The absolute thickness of the lubricity coating at single measurement points can be higher or lower than the range limits of the average thickness, with maximum deviations of preferably +/−50%, more preferably +/−25% and even more preferably +/−15% from the average thickness. However, it typically varies within the thickness ranges given for the average thickness in this description.

### Hollow Cathode Plasma

In certain embodiments, the generation of a uniform plasma throughout the portion of the vessel to be coated is contemplated, in particular when applying the barrier coating, as it has been found in certain instances to generate an SiOₓ coating providing a better barrier against oxygen. Uniform plasma means regular plasma that does not include a substantial amount of hollow cathode plasma (which has a higher emission intensity than regular plasma and is manifested as a localized area of higher intensity interrupting the more uniform intensity of the regular plasma).

The hollow cathode effect is generated by a pair of conductive surfaces opposing each other with the same negative potential with respect to a common anode. If the spacing is made (depending on the pressure and gas type) such that the space charge sheaths overlap, electrons start to oscillate between the reflecting potentials of the opposite wall sheaths leading to multiple collisions as the electrons are accelerated by the potential gradient across the sheath region. The electrons are confined in the space charge sheath overlap which results in very high ionization and high ion density plasmas. This phenomenon is described as the hollow cathode effect. Those skilled in the art are able to vary the processing conditions, such as the power level and the feed rates or pressure of the gases, to form uniform plasma throughout or to form plasma including various degrees of hollow cathode plasma.

### Moving Inlet in the First Phase

When the inlet is static during PECVD deposition process, the area close to the inlet (for example, the area close to the flange of a syringe) is coated thicker than the area distal to the inlet (for example, the area close to the needle and far from the flange in a syringe). Applicants have found that providing relative axial motion between the vessel and the gas inlet during the coating process allows the deposited lubricity layer to be more evenly distributed along the length of the vessel being coated.

### High Electromagnetic Power in the Second Phase

Low electromagnetic power may afford higher lubricity properties. However, it tends to give less adhered and less crosslinked coating which potentially leads to high particle count. Applicants found that increasing RF power results in a layer that more strongly adheres to the vessel and a high-power crosslinking treatment affords less particle counts.

The inventors also found that providing relative axial motion between the vessel and the gas inlet at the first phase and increasing the RF power at the second phase are able to reduce the number of particles liberated from the lubricity layer while maintain high lubricity properties. In sum, relative axial motion between the vessel and the gas inlet and/or low electromagnetic power applied during the first phase, combined with increasing RF power/or pressure at the second phase provides lubricity to a greater portion of the container, with less potential for particulate formation.

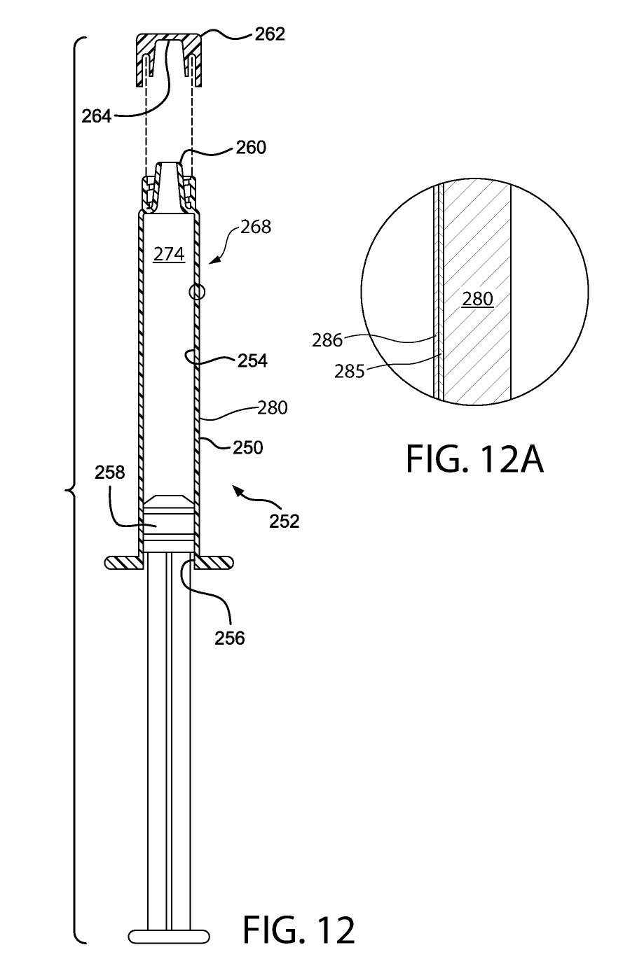

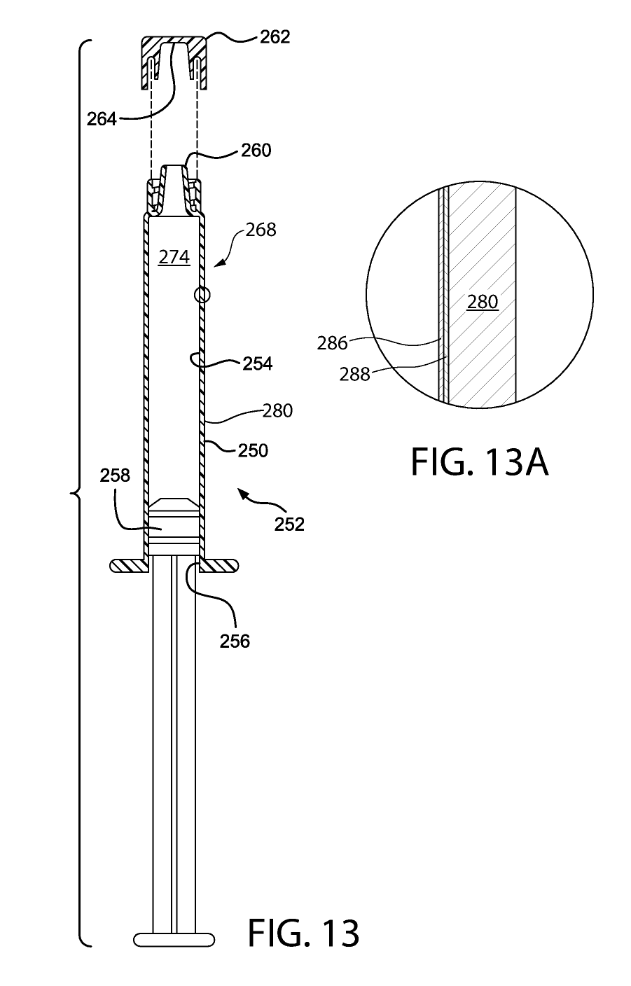

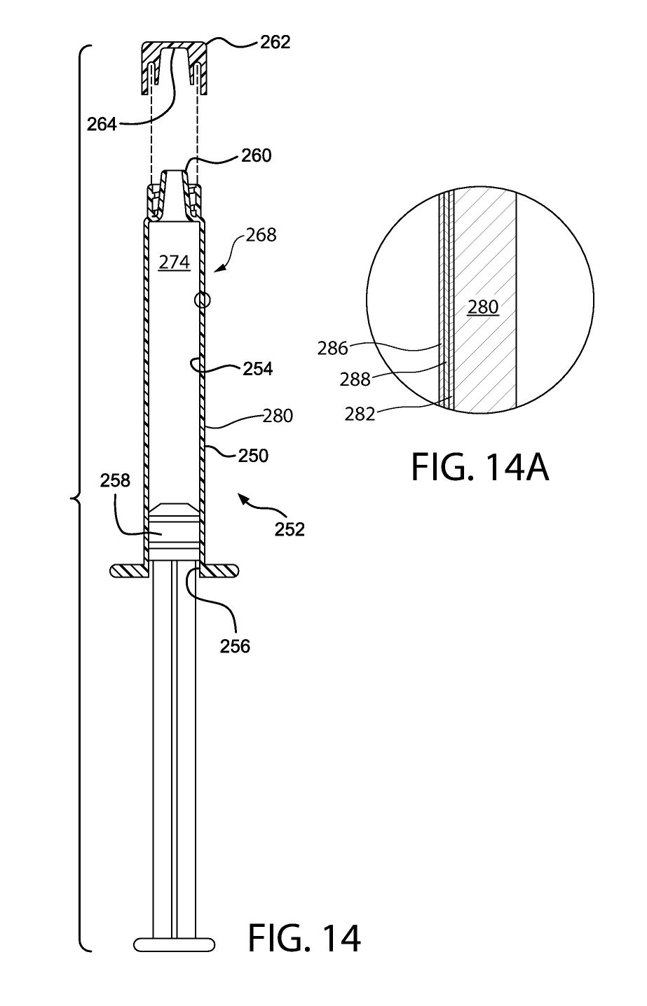

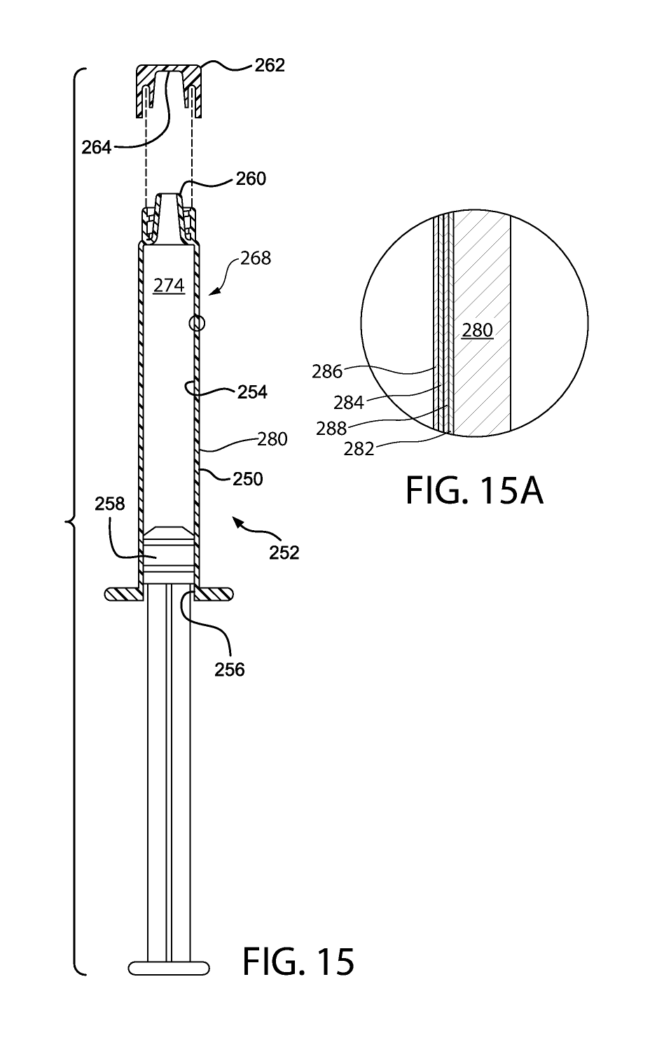

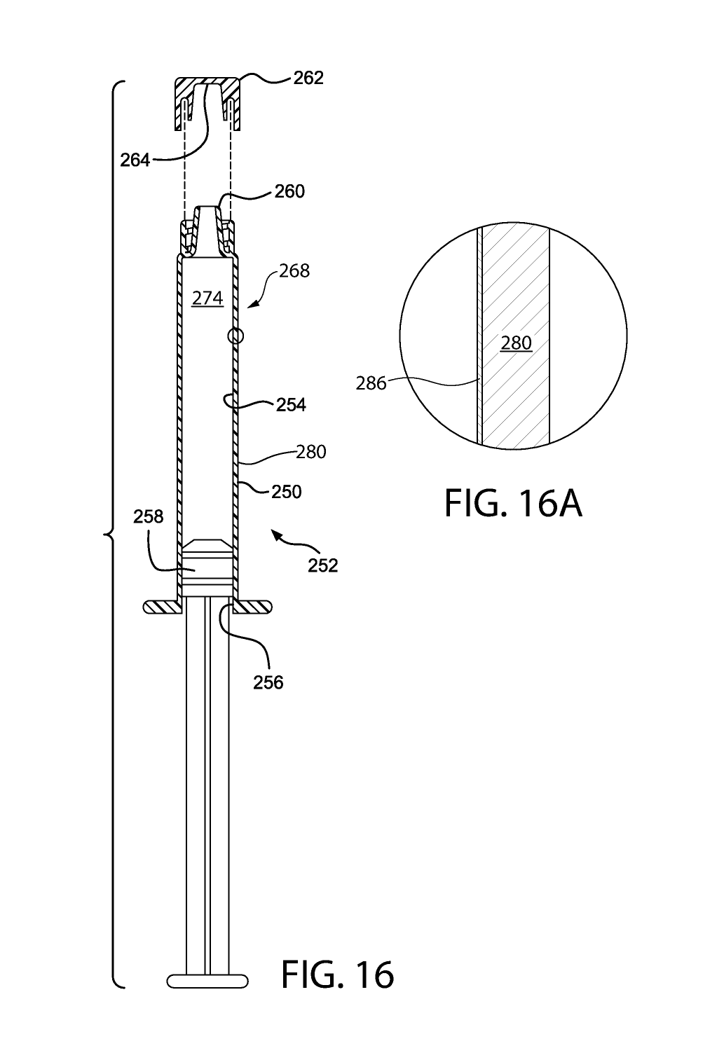

Referring to the drawings, a method for preparing a lubricity coating or layer 286 on a plastic substrate 280 such as the interior surface 254 of a vessel 268, 80, for example on its wall 280, is illustrated. One example of a suitable vessel, shown in FIGS. 12 and 12A, is a syringe barrel 250 for a medical syringe 252 having an inner surface 254. Such syringes 252 are sometimes supplied prefilled with saline solution, a pharmaceutical preparation, or the like for use in medical techniques.

A syringe barrel 250 as molded commonly can be open at both the back end 256, to receive a plunger 258, and at the front end 260, to receive a hypodermic needle, a nozzle, or tubing for dispensing the contents of the syringe 252 or for receiving material into the syringe 252. But the front end 260 can optionally be capped and the plunger 258 optionally can be fitted in place before the prefilled syringe 252 is used, closing the barrel 250 at both ends. A cap 262 can be installed either for the purpose of processing the syringe barrel 250 or assembled syringe, or to remain in place during storage of the prefilled syringe 252, up to the time the cap 262 is removed and (optionally) a hypodermic needle or other delivery conduit is fitted on the front end 260 to prepare the syringe 252 for use.

When a vessel 268 is coated by the above coating method using PECVD, the coating method comprises several steps. A vessel 268 is provided having an open end, a closed end, and an interior surface. At least one gaseous reactant is introduced within the vessel 268. Plasma is formed within the vessel 268 under conditions effective to form a reaction product of the reactant, i.e. a coating, on the interior surface of the vessel 268.

### Two-Phase OMCTS Lubricity Coating Process

### First Phase:

Apparatus and general conditions suitable for carrying out this step are described in U.S. Pat. No. 7,985,188 and EP2796591A1, which is incorporated here by reference in full except at least during the coating process, the gas inlet is moving axially. The electric power at this phase is the first power and the pressure at this phase is the first pressure. Exemplary coating parameters related to this step are shown below.

Coating Parameters at First Phase

| | | Plasma | Plasma | RF | RF | RF | | |
|---|---|---|---|---|---|---|---|---|
| | Plasma | Initial | Ramp | Wattage | Wattage | Wattage | RF NET | Chuck |
| | DELAY | Duration | Duration | Initial | Ramp | Ramp | POWER | Pressure |
| STEP | (ms) | (ms) | (ms) | (W) | Start (W) | End (W) | (W) | (Torr) |
| 1 | 5,000 | 0 | 0 | 0.0 | 0.0 | 0.0 | 0.0 | 0.15 |
| 2 | 0 | 2,000 | 500 | 50.0 | 15.0 | 8.0 | 22.0 | 0.15 |
| 3 | 200 | 0 | 1,000 | 0.0 | 0.0 | 0.0 | 0.0 | 0.15 |
| 4 | 0 | 100 | 3,000 | 5.0 | 5.0 | 10.0 | 5.0 | 0.15 |
| 5 | 200 | 0 | 1,000 | 0.0 | 0.0 | 0.0 | 0.0 | 0.15 |
| 6 | 0 | 100 | 3,000 | 5.0 | 5.0 | 10.0 | 5.0 | 0.15 |
| 7 | 200 | 0 | 1,000 | 0.0 | 0.0 | 0.0 | 0.0 | 0.15 |
| 8 | 0 | 100 | 3,000 | 5.0 | 5.0 | 10.0 | 5.0 | 0.15 |
| 9 | 200 | 0 | 1,000 | 0.0 | 0.0 | 0.0 | 0.0 | 0.15 |
| 10 | 0 | 100 | 3,000 | 5.0 | 5.0 | 10.0 | 5.0 | 0.15 |
| 11 | 200 | 0 | 1,000 | 0.0 | 0.0 | 0.0 | 0.0 | 0.15 |
| 12 | 0 | 100 | 3,000 | 5.0 | 5.0 | 10.0 | 5.0 | 0.15 |
| 13 | 200 | 0 | 1,000 | 0.0 | 0.0 | 0.0 | 0.0 | 0.15 |
| 14 | 0 | 100 | 3,000 | 5.0 | 5.0 | 10.0 | 5.0 | 0.15 |
| 15 | 200 | 0 | 1,000 | 0.0 | 0.0 | 0.0 | 0.0 | 0.15 |
| 16 | 0 | 100 | 3,000 | 5.0 | 5.0 | 10.0 | 5.0 | 0.15 |
| 17 | 200 | 0 | 1,000 | 0.0 | 0.0 | 0.0 | 0.0 | 0.15 |
| 18 | 0 | 100 | 3,000 | 5.0 | 5.0 | 10.0 | 5.0 | 0.15 |
| 19 | 200 | 0 | 1,000 | 0.0 | 0.0 | 0.0 | 0.0 | 0.15 |
| 20 | 0 | 100 | 3,000 | 5.0 | 5.0 | 10.0 | 5.0 | 0.15 |
| 21 | 200 | 0 | 1,000 | 0.0 | 0.0 | 0.0 | 0.0 | 0.15 |

| | OXYGEN | ARGON | MONOMER | | | | |
|---|---|---|---|---|---|---|---|
| | FLOW | FLOW | FLOW | PLASMA | DELAY | | |
| | RATE | RATE | RATE | HEIGHT | HEIGHT | | |
| STEP | (sccm) | (sccm) | (sccm) | (mm) | (mm) | Electrode | Coating |
| 1 | 0.0 | 0.0 | 20.0 | 40.00 | 40.00 | Circular | OMCTS |
| | | | | | | | Coating |
| 2 | 0.0 | 0.0 | 20.0 | −3.00 | 40.00 | Circular | OMCTS |
| | | | | | | | Coating |
| 3 | 0.0 | 0.0 | 20.0 | −3.00 | −20.00 | Circular | OMCTS |
| | | | | | | | Coating |
| 4 | 0.0 | 0.0 | 20.0 | 40.00 | −3.00 | Circular | OMCTS |
| | | | | | | | Coating |
| 5 | 0.0 | 0.0 | 20.0 | −3.00 | −20.00 | Circular | OMCTS |
| | | | | | | | Coating |
| 6 | 0.0 | 0.0 | 20.0 | 40.00 | −3.00 | Circular | OMCTS |
| | | | | | | | Coating |
| 7 | 0.0 | 0.0 | 20.0 | −3.00 | −20.00 | Circular | OMCTS |
| | | | | | | | Coating |
| 8 | 0.0 | 0.0 | 20.0 | 40.00 | −3.00 | Circular | OMCTS |
| | | | | | | | Coating |
| 9 | 0.0 | 0.0 | 20.0 | −3.00 | −20.00 | Circular | OMCTS |
| | | | | | | | Coating |
| 10 | 0.0 | 0.0 | 20.0 | 40.00 | −3.00 | Circular | OMCTS |
| | | | | | | | Coating |
| 11 | 0.0 | 0.0 | 20.0 | −3.00 | −20.00 | Circular | OMCTS |
| | | | | | | | Coating |
| 12 | 0.0 | 0.0 | 20.0 | 40.00 | −3.00 | Circular | OMCTS |
| | | | | | | | Coating |
| 13 | 0.0 | 0.0 | 20.0 | −3.00 | −20.00 | Circular | OMCTS |
| | | | | | | | Coating |
| 14 | 0.0 | 0.0 | 20.0 | 40.00 | −3.00 | Circular | OMCTS |
| | | | | | | | Coating |
| 15 | 0.0 | 0.0 | 20.0 | −3.00 | −20.00 | Circular | OMCTS |
| | | | | | | | Coating |
| 16 | 0.0 | 0.0 | 20.0 | 40.00 | −3.00 | Circular | OMCTS |
| | | | | | | | Coating |
| 17 | 0.0 | 0.0 | 20.0 | −3.00 | −20.00 | Circular | OMCTS |
| | | | | | | | Coating |
| 18 | 0.0 | 0.0 | 20.0 | 40.00 | −3.00 | Circular | OMCTS |
| | | | | | | | Coating |
| 19 | 0.0 | 0.0 | 20.0 | −3.00 | −20.00 | Circular | OMCTS |
| | | | | | | | Coating |
| 20 | 0.0 | 0.0 | 20.0 | 40.00 | −3.00 | Circular | OMCTS |
| | | | | | | | Coating |
| 21 | 0.0 | 0.0 | 20.0 | −3.00 | −20.00 | Circular | OMCTS |
| | | | | | | | Coating |

### Second Phase:

The electric power at this phase is the second power and the pressure at this phase is the second pressure. The second power is higher than the first power. Exemplary coating parameters related to this step are shown below.

Crosslinking Parameters at First Phase

| Plasma | Plasma Initial | RF Forward | RF Reflected | Plasma | Delay | |
|---|---|---|---|---|---|---|
| Delay (ms) | Duration (ms) | Power (Watts) | Power (Watts) | Height (mm) | Height (mm) | Gas |
| 5000 | 1000 | 50 | 10 | −20 | −20 | Air |

### Protocol for Plunger Force Testing

The testing can be carried out to measure the initial force Fi and the maintenance force Fm required to start and continue to move the plungers in the barrels of syringes. The plungers used for testing are Datwyler Omniflex or other suitable plungers. INSTRON compression testing instrument is used for the testing.

The testing procedures are as follows:

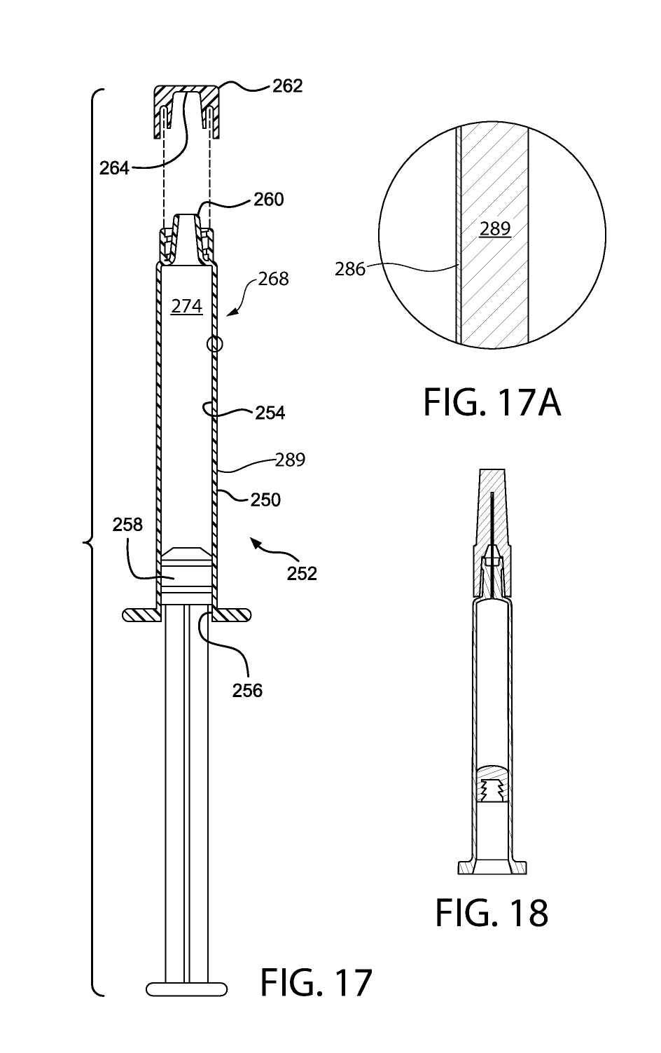

The plungers are inserted into the syringes obtained for testing using the plunger loading instrument. Plungers are inserted 18 mm into barrel (see FIG. 17) before testing, unless otherwise specified. The needle shields are removed prior to testing. The syringes with the plunger are loaded into the compression testing instrument to determine the Fi and Fm.

Follow the specific work instructions for operating the compression testing instrument. The starting distance between the cross head member of the Instron testing instrument and the end of the plunger rod is about 12.5 mm in Example 4. Choose the method in the system software specific to the samples being tested. Samples are tested at 300 mm/min. Export or save the data according to the instrument's work instructions.

### Protocol for Particle Count Testing

This protocol applies to the analysis of sub-visible particles in the coated device using Micro Flow Imaging (MFI, i.e. FlowCam).

Particle Free Water (PFW): Type I water (ultra-pure, with a resistivity of 18.2 MΩ) that has been filtered through a 0.22 μm pore-size filter can be obtained from a Millipore MilliQ or equivalent water filtration system.

Equipment:

Fluid Imaging Technologies FlowCam Particle Counter; Particle Free Water PFW (Millipore Milli-Q or equivalently filtered PFW); Laminar flow hood; 15 or 50 mL Polypropylene (PP) Sample Tubes with Caps or equivalent container; Calibrated pipette and disposable tips; Waste container; and NIST Traceable Particle Count Standard (Validex, Thermo or equivalent)

Procedures:

1. Start Up:

Ensure the laminar flow hood is on and has been for at least 0.5 hours before attempting to conduct particle analysis. Visually inspect the samples for visible particles in the laminar flow hood. The presence of a visible particle requires that a replacement sample be obtained for testing to prevent unnecessary clogging of the instrument. Obtain an empty 200 mL beaker or other appropriate container for waste and place the FlowCam waste line into the beaker. Perform instrument start-up verification checks as outlined in the instrument work instructions. Continue to follow these work instructions for instrument-specific guidelines to operating the software during the testing procedure outlined below.

2. Standard Container Analysis Run Conditions

Container analysis is performed using the 10× objective and 100 um Standard flow cell. The sample aspiration volume is pre-set to 0.95 mL. Verify that the 10× objective is installed and that the edges of the flow cell are aligned to vertical relative to the field of view and that the field of view is centered across the flow cell on the x-y axis. Confirm that the flow cell is clean and there are no contaminants on the outer surface that will effect calibration. If there are carefully clean the outside of the flow cell with a Windex wetted leans paper followed by dry lens paper. Select the 10×FC100 um 1mL.cxt context file (Default Context File) from the context tab in Visual Spreadsheet. In the Setup and Focus mode with the pump on at a flow rate of −0.2 mL/min and PFW moving through the flow cell, verify the Intensity Mean is reading 150 (+/−1). If an adjustment is required, make small changes to the flash duration in the Setup and Focus—Setup—Camera menu (typical set-point 20.1-20.5).

3. Setup and Focus

Open Setup and Focus tab and flush the system with a minimum of 10 volumes of PFW by adding PFW water using a transfer pipette directly into the aspiration funnel and push the button to start (resume) pump at a flow rate of not to exceed 0.5 mL/min. In Setup and focus mode set the flow rate of 0.1 mL/min aspirate either the 10 um NIST bead standard or a pre-shaken (reserved) sample pool containing 1-OMCTS/Si oil droplets starting and stopping the flow while simultaneously adjusting the fine focus knob on the front of the instrument as particles pass through the flow cell.

4. Blank Verification Analysis

Prepare as many PP sample tubes as required based on the number of samples to be analyzed and the pool size (typically 10 containers/pool) by rinsing the inside and outside of the tubes and the caps at least 5× with PFW water. Rinse the inside of each tube by adding approximately 10 ml of PFW, capping, vigorously shaking, and then emptying the rinsate into the sink and gently shake out all drops. Repeat this 5 more times. Fill and cap and place tubes in the laminar flow hood. Before starting the Blank Verification Test, flush the system three times with an aliquot from one of the prepared tubes in the Setup and Focus mode. Using a 5× pre-rinsed disposable transfer pipette aspirate ˜1.5 mL from one of the pre-prepared PP sample tubes and aspirate the liquid into the flow cell in the Setup and Focus Mode. Run the Blank Verification identifying the sample as 10× Start-up Blank Run 1.

Blank Verification Acceptance Criteria

| | 5 um | 10 um |
|---|---|---|
| Blank Specification | <50 | <10 |

If the blank fails to meet acceptance criteria flush the system with 10 additional volumes of PFW and repeat the Blank Verification Check.

5. Particle Count Standard—System Suitability Check

Before starting the System Suitability Check, flush the system with an aliquot of PFW in the Setup and Focus mode.
Mix the Counting Standard solution by gently inverting 10 times and decanting an amount suitable for testing into a pre-cleaned sample tube. Let the pooled sample stand for a minimum of 5 minutes to degas before testing. Using a pre-rinsed disposable transfer pipette fill the dispensing funnel directly with ˜1.5 mL of the Validex or Thermo 10× Counting Standard and aspirate in the Setup and Focus mode until the liquid volume enters the flow cell then exit Setup and Focus. Run the Counting Standard identifying the sample as 10× Start-up Particle Count Standard Run 1.

The particle count per mL should be within the specifications listed on the C of A/Label of the Counting Standard. Validex for example, should measure between 900 and 1100 particles/mL (1000 particles/mL+/−10%) in the >5 μm channel (Note the standard represents a small size range of reference beads and as long as the blank check passed acceptance criteria there is no reason to expect interference effecting the cumulative counts for the reference material).

Acceptance Criteria: +/−10% of the label claim.

6. Analysis of Syringes

Fill each syringe in the sample pool with the stated syringe volume of PFW and let them sit for 5 minutes before dispensing (e.g. 1 mL for 1 mL syringes). Pour the extraction volume from each syringe within the pool into a pre-labelled/pre-cleaned PP syringe using caution to avoid agitation. Allow the pool to sit for a minimum of 5 minutes to dissipate any suspended air bubbles generated by the transfer prior to aspiration. The minimum pool size for syringes is four 1 mL syringes/pool or 4 mL. This will allow for enough liquid volume for a single replicate injection. Run the sample pool Identifying the individual analysis with the sample information. After the run, be sure to save the run analysis summary (as a .pdf) in a network folder. Perform a single replicate Injection from the same pool comparing the Sum counts between the two injections from the same pool. These should compare within +/−10%. After the replicate run aspirate a single volume of PFW through the flow cell in Setup and Focus Mode and dispense to dryness. Perform analysis of each pool in duplicate (as above) followed by a single aspiration funnel volume of PFW water ˜2 mL until all sample pools and/or groups are analyzed. Transcribe a summary report of the results for each group of samples.

7. Analysis of Vials

Fill each container in the sample pool with the stated container volume of PFW and let them sit for 5 minutes before dispensing (e.g. 6 mL for 6 mL Vials). Hand seal and agitate the vials within the pool for 10 seconds and pour the extraction volume from each container within the pool into a pre-labelled/pre-cleaned PP container. Allow the pool to sit for a minimum of 5 minutes to dissipate any suspended air bubbles generated by the transfer prior to aspiration. The minimum pool size for vials is 6 mL (one 6 mL or 10 mL vial/pool or three 2 mL vials). This will allow for enough liquid volume for a single replicate injection. Run the sample pool Identifying the sample analysis with the sample information e.g. DHR2016-365-0078 Pool 1 Run 1. After the run, be sure to save the run analysis summary (as a .pdf) in a network folder. Perform a single replicate Injection from the same pool comparing the 5 um counts between the two injections. These should compare within +/−20%. After the replicate run aspirate a single volume of PFW through the flow cell in Setup and Focus Mode and dispense the funnel to dryness. Perform analysis of each subsequent pool in duplicate as above followed by a single aspiration funnel volume of PFW water ˜2 mL until all sample pools and/or groups are analyzed. Transcribe a summary report for each group of samples.

8. Alternate Diluents/Buffers

The test protocol may require extraction with a solution other than PFW. The testing solution must be filtered through 0.22 micron pore size filter and tested before use to ensure the particle counts does not exceed the parameters outlined above. Document sample preparation and testing on the designated form.

Preparation of 1 L citrate buffer solution (pH=3.5):

Place a 1000 mL beaker and stir bar on balance and tare. Weigh out 5.88g+/−0.01 g of sodium citrate dehydrate and add to beaker. Add approximately 700 mL of HPW. Stir until completely dissolved. Adjust pH to 3.5+/−0.1 with 2% HNO3. QS to 1000g+/−5.0 g with HPW.

If testing fewer than 10 samples, run another particle count standard at the end of testing. If testing more than 10 samples, the suggested sample sequence is:
1) Environment Test Blank; 2) Particle Count Standard; 3) 10 samples (and associated blanks for filling test articles); 4) Particle Count Standard; Repeat 1) and 4) as necessary until all samples are tested.

Unless specified in the protocol, no sample results will be corrected for blank particle counts. Results are typically reported as either Particles/mL or Particles/Container. Depending on the protocol or specification, summary reports must clearly identify which. Unless otherwise specified, cumulative particle counts results are rounded up to the next whole number and reported as particles/mL for each size channel. Specifications are established for empty containers and should not be applied to filled, stoppered presentations.

Although the invention is not limited according to the accuracy or applicability of this theory of operation

### EXAMPLES

The invention will now be illustrated in more detail with reference to the following Examples, but it should be understood that the present invention is not deemed to be limited thereto.

### Example 1—Increase of Phase 2 Power

To decrease the number of sub-visible particles produced by the lubricity coating in an aqueous solution-filled syringe, phase 2 power was increased. Tables 1 and 2 respectively describe coating settings used in this example and their effects.

Table 1 describes the coating settings used to produce particle count results in Table 2. Table 2 provides particle count data from a FlowCAM. The syringes were filled with 1 mL of filtered water and shaken horizontally by hand for 10 seconds. The liquid was removed using a pipette and dispensed into the FlowCAM. Table 2 demonstrates that increasing the phase 2 power during the PECVD process significantly reduces sub-visible particle release from the lubricity layer into the liquid contents of a syringe.

TABLE 1

| | | Oxygen | | Argon | | |
|---|---|---|---|---|---|---|
| | | Flow | OMCTS | Flow | Phase 2 | Phase 2 |
| | | Rate | Flow Rate | Rate | Power | Duration |
| Coating | Inlet | (sccm) | (sccm) | (sccm) | (W) | (sec) |
| Low Power | ⅛″ | 3.1 | 4 | 7.5 | 2 | 15 |
| High Power | ⅛″ | 3.1 | 4 | 7.5 | 11 | 15 |

TABLE 2

| | Syringe shaken by hand for 10 seconds, | | | | | |
|---|---|---|---|---|---|---|
| | solution pipetted from syringe | | | | | |
| | >1 μm | | >10 μm | | >25 μm | |
| Coating | Mean | 2σ | Mean | 2σ | Mean | 2σ |
| Low Power | 253,157 | 97,685 | 4,918 | 2,311 | 93 | 58 |
| High Power | 63,380 | 37,409 | 1,169 | 899 | 1 | 4 |

As Table 2 illustrates, where the phase 2 power was high (e.g., 11 W) instead of low (e.g., 2 W), the sub-visible particulate count was significantly less. This indicates that using high phase 2 power adheres the lubricity layer to the vessel better than low phase 2 power.

### Example 2—Stationary Inlet Vs. Moving Inlet

When testing syringes that had been coated with higher-than-normal phase 2 power, it was observed that the lubricious properties of the lubricity layers were better at positions closer to the flanges of the syringes. It was found that the coating near the flange is much thicker than the area close to the distal end (need end). Sometimes the coating near the distal end is too thin (e.g. under 50 nm) to be considered acceptable. The inventors surmised this was caused by the stillness of the gas inlet since during the coating, the inlet was closer to the flange than to the distal end. The inventors believe that if such uniformity would be improved, then the plunger force would improve. The inventors successfully achieved these goals by axially moving the inlet within the syringe. The gas inlet was moving vertically from the bottom (i.e. the flange side) to the top (i.e. the needle end) of the coated article. The rate of vertical movement of the inlet is determined by 1) the Length of which the inlet needs to travel and 2) the plasma ramp duration. In this study, the inlet was moving at a rate of 14.3 mm/sec. Table 3 and FIG. 3 respectively describe coating settings and related results for stationary inlets versus axially moving inlets.

TABLE 3

| | | Oxygen | | Argon | | |
|---|---|---|---|---|---|---|
| | | Flow | OMCTS | Flow | Phase 2 | Phase 2 |
| | | Rate | Flow Rate | Rate | Power | Duration |
| Coating | Inlet | (sccm) | (sccm) | (sccm) | (W) | (sec) |
| Stationary | ⅛″ | 3.1 | 4 | 7.5 | 10 | 15 |
| Moving | Moving | 4 | 4 | 7.5 | 12 | 15 |
| | ⅛″ | | | | | |

FIG. 3 is a graph illustrating the effect of a moving gas inlet on high power OMCTS coating using the coating settings described in Table 3. FIG. 3 shows that moving the gas inlet during PECVD results in substantially uniform and low plunger sliding force compared to syringes in which the lubricity layer was applied while the inlet was stationary.

### Example 3—Stationary Inlet Low Power Vs. Moving Inlet High Power

Table 4, below, describes coating settings used to produce particle count data presented in Tables 5 and 6, below.

TABLE 4

| | | Oxygen | | Argon | | |
|---|---|---|---|---|---|---|
| | | Flow | OMCTS | Flow | Phase 2 | Phase 2 |
| | | Rate | Rate | Flow Rate | Power | Duration |
| Coating | Inlet | (sccm) | (sccm) | (sccm) | (W) | (sec) |
| A | ⅛″ | 3.1 | 4 | 7.5 | 2 | 15 |
| B | Moving | 4 | 4 | 8 | 12 | 15 |
| | ⅛″ | | | | | |

Table 5 shows particle count data from a FlowCAM after a relatively gentle preparation in which the syringes were filled with 1 mL of filtered water and inverted 20 times by hand. The liquid was removed using a pipette and dispensed into the FlowCAM.

TABLE 5

| | Syringe inverted 20 times, solution pipetted from syringe | | | | | | | | | |
|---|---|---|---|---|---|---|---|---|---|---|
| | >1 μm | | >2 μm | | >5 μm | | >10 μm | | >25 μm | |
| Coating | Mean | 2σ | Mean | 2σ | Mean | 2σ | Mean | 2σ | Mean | 2σ |
| A | 9,769 | 5,418 | 5,815 | 3,373 | 1,420 | 982 | 204 | 133 | 1 | 4 |
| B | 2,005 | 1,593 | 921 | 760 | 139 | 173 | 16 | 19 | 1 | 4 |

Table 6 shows particle count data from a FlowCAM after a relatively aggressive preparation in which the syringes were filled with 1 mL of filtered water, placed horizontally on a shaker table and shaken for 10 minutes at 1000 RPM. The liquid was then expressed through the needle into the FlowCAM.

TABLE 6

| | Horizontal for 10 min on a shaker table at 1000 RPM, solution expressed through needle | | | | | | | | | |
|---|---|---|---|---|---|---|---|---|---|---|
| | >1 μm | | >2 μm | | >5 μm | | >10 μm | | >25 μm | |
| Coating | Mean | 2σ | Mean | 2σ | Mean | 2σ | Mean | 2σ | Mean | 2σ |
| A | 218,446 | 175,853 | 112,741 | 94,771 | 27,928 | 25,956 | 4,243 | 4,866 | 47 | 49 |
| B | 47,027 | 161,744 | 22,717 | 83,315 | 2,651 | 10,842 | 119 | 524 | 1 | 4 |

Together, Tables 5 and 6 show that particle count is dramatically reduced when the gas inlet is moved axially during PECVD and when phase 2 power is high (e.g., 12 W).

While examples described herein involved moving the gas inlet, it is contemplated that similar results may be achieved by axially moving the vessel (e.g., syringe) during PECVD instead of, or in addition to, moving the inlet. Thus, the invention broadly relates to providing relative axial motion between the vessel and the gas inlet during PECVD application of a lubricity layer. More preferably, such relative motion is provided during the first phase.

### Example 4—Moving Inlet at 1^{st} Phase Plus High Power Crosslinking at 2^{nd} Phase

The purpose of this study is to evaluate four processes regarding the resulting plunger force and particle count.

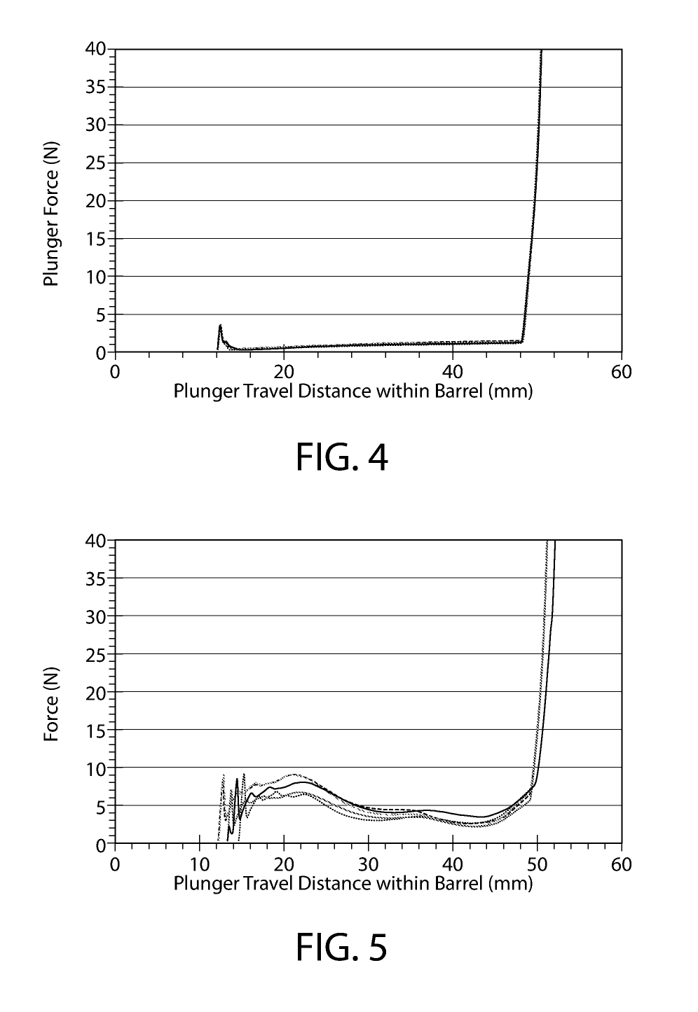

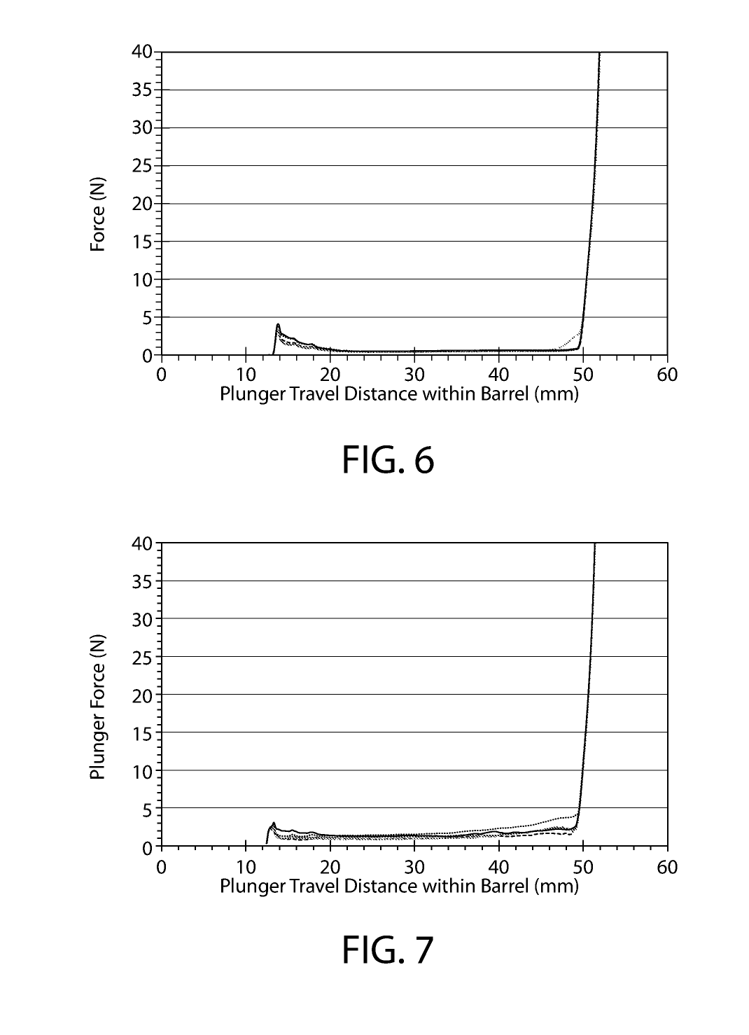

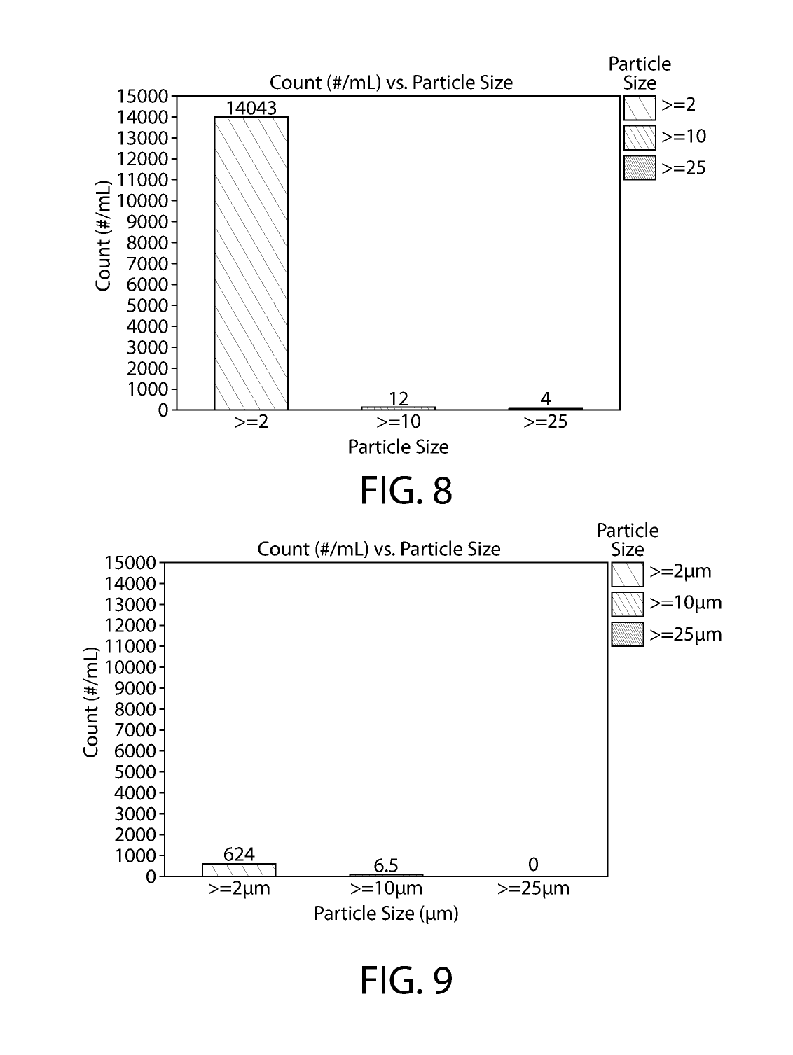

Process 1 was a one phase lubricity coating process with low power and static inlet. Process 2 was a one phase lubricity coating process with high power and moving inlet and Process 3 was a two-phase lubricity coating process with low power and moving inlet at the first phase, followed by a post crosslinking step of a moderately higher power (e.g. 10 W) at the second phase. Process 4 was also a two-phase lubricity coating process with low power and moving inlet at the first phase, followed by a post crosslinking step of high power (e.g. 50 W) at the second phase. At the second phase, the closure (if applicable, e.g. syringe cap) was removed, the coated surface was crosslinked using plasma, with air at the atmospheric pressure, at the second electric power level and the second pressure. No organosiloxane gas monomer was used at the second phase of processes 3 and 4, and the inlet was kept static at the starting position. The power level of the second power was higher than the power level of the first power. Table 7 below, describes coating settings used to produce the plunger forces shown in FIGS. 4, 5, 6 and 7. The particle count data for processes 3 and 4 which were obtained using citrate buffer according to the “Protocol for Particle Count Testing” in the specification, are presented in Table 8 and FIGS. 8 and 9.

TABLE 7

| | | | | | | Oxygen | Argon |
|---|---|---|---|---|---|---|---|
| | | | | | OMCTS | Flow | Flow |
| Proc- | | | Power | Pressure | Flow Rate | Rate | Rate |
| ess | Phase | Inlet | (W) | (torr) | (sccm) | (sccm) | (sccm) |
| 1 | 1 | Static | 1 | 1.5 | 4 | 3 | 7.5 |
| 2 | 1 | Moving | 1-9 | 1.5 | 4 | 3 | 7.5 |
| 3 | 1 | Moving | 1 | 1.5 | 5 | 2 | 6 |
| | 2 | Static | 10 | 2.1 | 0 | 0 | 0 |
| 4 | 1 | Moving | 5-10 | 0.2 | 20 | 0 | 0 |
| | 2 | Static | 50 | 2.1 | 0 | 0 | 0 |

The Processes 1 and 2 were both one phase coating processes while Process 1 employed static inlet with lower power and Process 2 employed moving inlet with higher power. The results show that Process 1 afforded low plunger force but high particle count while Process 2 afforded higher plunger force with lower particle counts. It is contemplated that lower power leads to lower crosslinking density which gives lower plunger force and higher particle counts. While lower plunger force is desired, high particle count is undesirable. How to balance the two conflicting requirements is a long-standing need in the industry.

Processes 3 and 4 were both two-phase coating processes, intended to provide a solution to the need by achieving the combination of low plunger force and low particle count. The inventors surmised that moving inlet and low power applied at the 1^{st} phase gives a uniform lubricity coating and low plunger force and the subsequent high power post-treatment, a crosslinking step, increases crosslinking density to produce low particle count. The main difference between Processes 3 and 4 is the power used at the 2^{nd} phase. The power at the 2^{nd} crosslinking phase was dramatically increased from 10 w for Process 3 to 50 w for Process 4. The particle count data obtained by FlowCam show that the particle count was dramatically lowered for Process 4 compared to the rest processes 1-3. The most obvious improvement of Process 4 over Process 3 is the substantial decrease of particle count from 14043 (>2 um) to 624 (>2 um) while keeping comparable plunger force. Therefore, the combination of low plunger force and low particle count was obtained through a two-phase lubricity coating process with moving inlet at the first phase and high power post crosslinking at the second phase.

TABLE 8
Particulate Count for all Processes

| | Particle Size (μm) | | |
|---|---|---|---|
| Process Number | 2 | 10 | 25 |
| Process 1 | 8400 | 1156 | 35 |
| Process 2 | 4146 | 16 | 1 |
| Process 3 | 14043 | 12 | 4 |
| Process 4 | 624 | 7 | 0 |

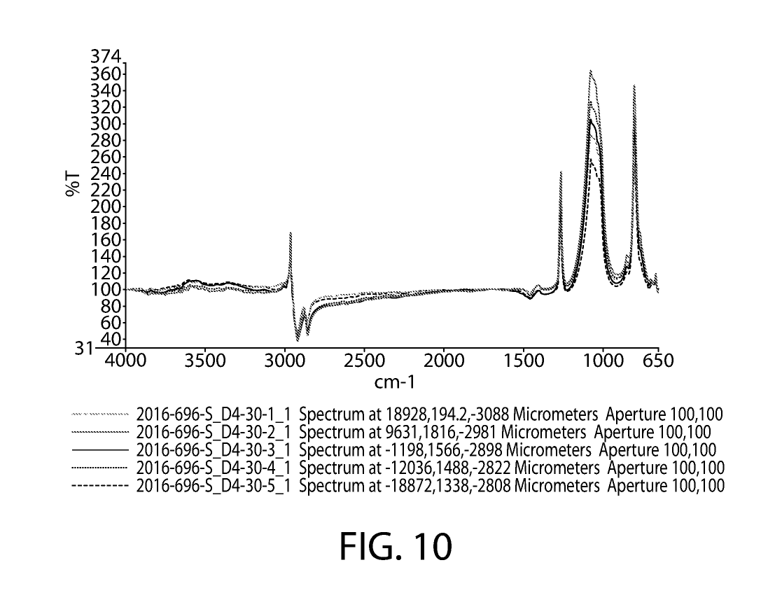

The FT-IR spectrum for the coating prepared by Process 4 is shown in FIG. 10.

Table 9 presents the thickness profile data of the coatings generated by Process 1 and Process 4 which exhibit the uniformity of the coatings. The results show that Process 4 affords more uniform lubricity coating along the entire barrel length, from the proximal end to the distal end. Compared to Process 3.

As shown in Table 9, Process 4 affords a coating with narrower thickness range (Max-Min=250 nm) compared to Process 1 (Max-Min=400 nm). The standard deviation for Process 4 is also smaller than Process 1 (71 vs 160). These data demonstrate that Process 4 affords a more uniform lubricity coating which is consistent with the improved plunge force profile for Process 4 shown in FIG. 7.

TABLE 9
Thickness Profile by Filmetrics for Process 1 and Process 4

| | | Coating Thickness (nm) | |
|---|---|---|---|
| | Syringe Barrel | L-OMCTS | L-OMCTS |
| — | Location (mm) | (Process 1) | (Process 4) |
| Flange End (Proximal) | 5 | 400 | 400 |
| | 10 | 400 | 400 |
| | 15 | 350 | 450 |
| | 20 | 300 | 450 |
| | 25 | 200 | 500 |
| | 30 | 150 | 450 |
| | 35 | 100 | 400 |
| | 40 | 40 | 350 |
| | 45 | 0 | 350 |
| Needle End (Distal) | 50 | 0 | 250 |
| | Mean Thickness | 194 | 400 |
| | Std Dev. | 160 | 71 |
| | Min | 0 | 250 |
| | Max | 400 | 500 |

Height is the distance of the position from the flange.

This study was performed on a 4-Up coating platform. A 1-Up platform is a PECVD station where vacuum supply, process gases, and RF power are supplied to one individual article to deposit the coating or coating set on the surface of the coated article. A 4-Up platform is a similar station but in 4 configuration, where the vacuum supply, process gases, and RF power are uniformly split so that each are supplied uniformly to four individual articles. By maintaining uniformity with vacuum supply and process gases, the pressure within each article of a 4-Up remains the same as the single article of a 1-Up. Therefore, only power needs to be adjusted to compensate for the increased number of articles. Similarly, the coating system can be a multiple-Up platform, such as 8-Up, 16-Up, 32-Up etc. Multiple-up coating platform affords economic and other benefits.

While the invention has been described in detail and with reference to specific examples thereof, it will be apparent to one skilled in the art that various changes and modifications can be made therein without departing from the spirit and scope thereof.

What is claimed is:

1.-31. (canceled)

32. A method for applying a lubricity layer to an inner surface of a vessel, the method comprising:
1. providing the vessel to be processed including the inner surface defining a lumen and an opening at an end of the vessel providing access to the lumen;
2. providing a PECVD platform comprising:
a. a gas inlet having an internal passage having at least one outlet,
b. an outer electrode, and
c. a vessel port configured to receive and seat the opening of the vessel;
3. introducing a gaseous PECVD organosiloxane precursor into the lumen via at least one outlet of the internal passage;
4. applying electromagnetic energy to the outer electrode under conditions effective to form a PECVD lubricity layer on at least a portion of the inner surface of the vessel; and
5. providing a relative motion, optionally axial, between the vessel and the gas inlet during at least some time when electromagnetic energy is applied to the outer electrode.

33. A method for applying a lubricity layer to an outer surface of an object, the method comprising:
1. providing an object with an outer surface;
2. providing a PECVD platform comprising:
a. a gas inlet having an internal passage having at least one outlet,
b. an outer electrode, and
c. an object holder to hold the object;
3. introducing a gaseous PECVD organosiloxane precursor into a chamber around the object;
4. applying electromagnetic energy to the outer electrode under conditions effective to form a PECVD lubricity layer on at least a portion of the outer surface of the object; and
5. providing a relative motion, optionally axial, between the object and the gas inlet during at least some time when electromagnetic energy is applied to the outer electrode.

34. The method of claim 32, wherein the electromagnetic energy is applied to the outer electrode during a first phase and then subsequently a second phase, wherein the power level of the electromagnetic energy applied during the second phase is higher than the power level of the electromagnetic energy applied during the first phase.

35. The method of claim 34, wherein a first gas is introduced at the first phase and a second gas is introduced at the second phase.

36. The method of claim 35, wherein the second gas comprises air, oxygen, nitrogen, carbon dioxide, ozone, hydrogen, hydrogen peroxide vapor, water vapor, any noble gas, or any combination of two or more of the above; and the second gas is essentially free of organosiloxane precursors.

37. The method of claim 36, wherein the second gas comprises air or argon or both.

38. The method of claim 37, wherein the second gas comprises atmospheric air.

39. The method of claim 32, wherein the plasma is characterized as hollow cathode plasma.

40. The method of claim 32, wherein the gaseous PECVD precursor comprises organosiloxane.

41. The method of claim 40, wherein the organosilicon is selected from the group consisting of a linear siloxane, a monocyclic siloxane, a polycyclic siloxane, a polysilsesquioxane, an alkyl trimethoxysilane, a linear silazane, a monocyclic silazane, a polycyclic silazane, a polysilsesquiazane, and a combination of any two or more of these precursors.

42. The method of claim 40, wherein the gaseous PECVD precursor comprises OMCTS.

43. The method of claim 32, wherein said inner surface is glass; and the lubricity layer is applied directly on said inner surface.

44. The method of claim 32, said inner surface is made of a polymer; and the lubricity layer is applied directly on said inner surface.

45. The method of claim 32, the lubricity layer is applied on said inner surface using organosiloxane as the gas precursor.

46. The method of claim 32, further comprising applying a coating set on the substrate before the lubricity coating is applied, the coating set comprising at least one of:
a. a tie coating or layer comprising SiOₓC_{y} or SiNₓC_{y} wherein x is from about 0.5 to about 2.4 and y is from about 0.6 to about 3, the tie coating or layer having an interior surface facing the lumen and an outer surface facing the wall interior surface;
b. a barrier coating or layer being from 2 to 1000 nm thick and comprising SiOₓ, wherein x is from 1.5 to 2.9, the barrier coating or layer of SiOₓ having an interior surface facing the lumen and an outer surface facing the interior surface of the tie coating or layer, the barrier coating or layer being effective to reduce the ingress of atmospheric gas into the lumen compared to a vessel without a barrier coating or layer;
c. a pH protective coating or layer comprising SiOₓC_{y} or SiNₓC_{y} wherein x is from about 0.5 to about 2.4 and y is from about 0.6 to about 3, the pH protective coating or layer having an interior surface facing the lumen and an outer surface facing the interior surface of the barrier coating or layer;

47. The method according to claim 46, in which a barrier coating is applied on the substrate by:
(a) providing a gas comprising an organosilicon precursor and an oxidizing gas, optionally O₂, in the vicinity of the substrate surface; and
(b) generating plasma in the vicinity of the substrate surface, thus forming an SiOₓ barrier coating, in which x is from 1.5 to 2.9 as measured by XPS, on the substrate surface by plasma enhanced chemical vapor deposition (PECVD).

48. The method of claim 32, wherein the substrate is a polymer selected from the group consisting of a polycarbonate, an olefin polymer, a cyclic olefin copolymer (COC), a cyclic olefin polymer (COP), and a polyester.

49. The method of claim 32, wherein a moving gas inlet is applied during the first phase, which affords a lower plunger initial force Fi or a low plunger maintenance force Fm, or both, for a surface compared to the plunger forces obtained for the surface coated by the same process except not providing relative axial motion between the vessel and the gas inlet during at any time when electromagnetic energy is applied to the outer electrode.

50. The method of claim 32, wherein the lubricity layer obtained is represented by a formula Si_{w}OₓC_{y}H_{z} or Si_{w}(NH)ₓC_{y}H_{z} wherein w is 1 as measured by X-ray photoelectron spectroscopy (XPS), x is from about 0.5 to about 2.4 as measured by XPS, y is from about 0.6 to about 3 as measured by XPS, and z is from 2 to about 9 as measured by at least one of Rutherford backscattering spectrometry (RBS) or hydrogen forward scattering (HFS).

51. A vessel comprising a lubricity layer on an inner surface of the vessel, wherein the lubricity layer is applied using the method comprising the steps of:
1) providing the vessel to be processed with the inner surface, wherein the inner surface defines a lumen and an opening at an end of the vessel providing access to the lumen;
2) providing a PECVD platform comprising:
a. a gas inlet having an internal passage having at least one outlet,
b. an outer electrode, and
c. a vessel port configured to receive and seat the opening of the vessel,
3) introducing a gaseous PECVD organosiloxane precursor into the lumen via at least one outlet of the internal passage;
4) applying electromagnetic energy to the outer electrode under conditions effective to form a PECVD lubricity layer on at least a portion of the inner surface of the vessel; and
5) providing a relative motion, optionally axial, between the vessel and the gas inlet during at least some time when electromagnetic energy is applied to the outer electrode.
